(19) United States
(12) Patent Application Publication
(10) Pub. No.: US 2026/0207244 A1
(43) Pub. Date: Jul. 23, 2026

(54) CRYOABLATION SYSTEM AND METHOD

(71) Applicant: CRYO MEDICA INC., Gainesville, FL (US)

(72) Inventors: André Jacques Du Plessis, Cape Town (ZA); Johannes Petrus Jordaan, Cape Town (ZA); Johannes Michiel Bredenkamp, Cape Town (ZA)

(21) Appl. No.: 19/142,470

(22) PCT Filed: Dec. 22, 2023
(86) PCT No.: PCT/IB2023/063182

§ 371 Date: Jun. 23, 2025

Related U.S. Application Data
(60) Provisional application No. 63/476,965, filed on Dec. 23, 2022.

(51) Int. Cl. A61B 18/02; A61B 17/00; A61B 18/00; A61B 18/04

(52) U.S. Cl. CPC A61B 18/02; A61B 2017/00199; A61B 2018/00577; A61B 2018/00744; A61B 2018/0262; A61B 2018/0293; A61B 2018/044

(57) ABSTRACT

There is provided a cryoablation system and method. The system may include a probe which has a hollow body that defines an inlet and an outlet, the inlet configured to receive a thermal fluid for circulation through the hollow body, and the outlet configured to discharge the thermal fluid from the hollow body. A fluid supply console may be remote from the probe and in fluid communication with the inlet of the probe. The fluid supply console may be arranged, in use, to provide the thermal fluid to the probe to enable heating or cooling by the probe. The fluid supply console may include a heating element configured to selectively heat the thermal fluid before it is provided to the inlet of the probe so as to enable selective heating of the probe.

### CROSS-REFERENCE TO RELATED APPLICATION

This application claims priority from U.S. provisional patent application number 63/476,965 filed on 23 Dec. 2022, which is incorporated by reference herein.

### FIELD OF THE PRESENT DISCLOSURE

The present disclosure relates to systems methods and apparatuses for cryoablation treatment, or systems and methods that use cold temperatures to destroy unwanted tissue.

### BACKGROUND TO THE PRESENT DISCLOSURE

Cryoablation is performed by inserting cryoprobe needles into malignant tissue, usually under imaging guidance. After inserting one or more cryoprobe needles into the target tissue, the cryoprobe is rapidly cooled, removing heat from the tissue by conduction via physical contact with the cryoprobe. Rapid cooling of the cryoprobe takes place by means of the Joule-Thompson effect, whereby rapid expansion of a gas results in a change in the temperature of the gas. Argon gas exhibits Joule-Thompson cooling when rapidly expanded at room temperature.

When high-pressure Argon gas reaches a distal aspect of the cryoprobe, the argon is forced through a narrow orifice and then allowed to rapidly expand to atmospheric pressure. The rapid expansion of the argon causes a decrease in the temperature of the gas (the Joule-Thompson effect), which is rapidly transferred by convection and conduction to the metallic walls of the cryoprobe. The depressurized gas is then vented back out of the cryoprobe.

Rapid cooling of the cryoprobe or needle may be efficient at destroying adjacent tissue, but sometimes the probe needs to be removed quickly, for example when medical circumstances of the patient require it. The frozen probe may be difficult or impossible to remove quickly without injuring the patient. Known cryoprobes may also be clunky, cumbersome and inefficient, or overly expensive.

The applicant considers there to be room for improvement.

The preceding discussion of the background to the present disclosure is intended only to facilitate an understanding thereof. It should be appreciated that the discussion is not an acknowledgment or admission that any of the material referred to was part of the common general knowledge in the art as at the priority date of the application.

### SUMMARY OF THE PRESENT DISCLOSURE

In accordance with an aspect of the present disclosure there is provided a cryoablation system comprising:
a probe which has a hollow body that defines an inlet and an outlet, the inlet configured to receive a thermal fluid for circulation through the hollow body, and the outlet configured to discharge the thermal fluid from the hollow body; and
a fluid supply console which is remote from the probe and in fluid communication with the inlet of the probe, wherein the fluid supply console is arranged, in use, to provide the thermal fluid to the probe to enable heating or cooling by the probe, and wherein the fluid supply console includes a heating element which is configured to selectively heat the thermal fluid before it is provided to the inlet of the probe so as to enable selective heating of the probe in use.

The system may include a controller having a processor and a memory, said memory containing instructions executable by said processor, to execute functions of one or more components of the system. The controller may, for example, form part of or be housed by the fluid supply console. The fluid supply console may include a valve system and/or one or more valve(s) and/or at least one valve which may be operable and/or selectable between a first state and a second state. In the first state, the valve may be configured to provide the thermal fluid to the probe, e.g., for cooling. In the second state, the valve may be configured to provide the thermal fluid which may be heated by the heating element to the probe.

The fluid supply console may be operable to automatically select the first state and/or the second state of the valve and/or valve system so as to selectively provide heating or cooling of the probe in use, for example upon receiving input from a user which is indicative of heating or cooling being required by the probe.

The controller may be operable to control the heating element so as to selectively heat the thermal fluid.

The fluid supply console may be arranged to provide a single type of fluid as thermal fluid to the probe to facilitate cooling by the probe in use, for example by cryoablation cooling and/or by freezing the probe.

The heating element may be arranged to selectively heat the single type of thermal fluid before it is provided to the probe, so as to selectively heat or thaw the probe body, or so as to heat the probe body in order to thaw or heat biological material or tissue that surrounds it.

The fluid supply console may be arranged to selectively provide one or more thermal fluids to the probe. For example, the fluid supply console may be arranged to selectively provide a first thermal fluid for cooling the probe, and a second thermal fluid for heating the probe.

The heating element of the fluid supply console may be arranged to selectively heat the second thermal fluid before it is provided to the inlet of the probe, for example to thaw the probe, or so as to heat the probe body in order to thaw or heat biological material or tissue that surrounds it.

The fluid supply console may include a valve or valve system which is arranged to selectively provide the first thermal fluid for operatively cooling the probe. Additionally, or alternatively, the valve or valve system may be arranged to selectively provide a second thermal fluid for operatively heating the probe.

The first thermal fluid may be a fluid selected so as to provide cooling by way of the Joule-Thomson effect, or to provide cooling in another way.

The second thermal fluid may be a fluid selected so as to provide heating by way of thermal conduction or thermal convection.

The first fluid and/or the second fluid may be selected so as to be thermally conductive.

The first thermal fluid may be argon and the second thermal fluid may be argon. Alternatively, the first thermal fluid may be argon and the second thermal fluid is air. Further alternatively, the first thermal fluid may be air and the second thermal fluid may be argon.

The thermal fluid or thermal fluids may be selected from a list including: argon or argon based fluids, air, helium or helium based fluids, nitrogen or nitrogen based fluids, liquid nitrogen, inert or ideal gases, or any other gas or liquid capable of thermally heating or cooling the probe, as the case may be.

The fluid supply console may be connected to a pressurized thermal fluid supply, and/or it may be connected to one or more pressurized thermal fluid supplies or supply reservoir(s). For example, a first reservoir for pressurized argon may be provided and in fluid communication with the fluid supply console; and/or a second reservoir for pressurized air may be provided and in fluid communication with the fluid supply console.

The present disclosure extends to a fluid supply console for use in a cryoablation system as defined above.

The present disclosure further extends to a probe for use in a cryoablation system as defined above.

The probe may have a hollow body which extends from a proximal end of the hollow body to a distal end thereof. The probe may include a fluid conveying tube extending inside the hollow body from the proximal end toward the distal end and which opens into the hollow body. The distal end of the hollow body may be closed so that thermal fluid is enabled to enter the hollow body through the fluid conveying tube and to heat or cool the body of the probe, as the case may be. For example, the thermal fluid may cool the probe body due to the Joule-Thomson (JT) effect. In other words, the probe may be arranged to be cooled due to an expansion in volume of the thermal fluid when the thermal fluid exits the fluid conveying tube inside the hollow body of the probe. The probe body may be heated so as to thaw or heat biological material or tissue that surrounds it which was frozen due to the aforementioned cooling by the JT effect.

The distal end of the probe body may be arranged to be inserted into a patient, e.g., for cryoablation treatment or cryo-treatment. The probe body may also be termed a needle body.

In accordance with another aspect of the present disclosure there is provided a cryoablation system comprising: a fluid supply console which is remotely connectable to a probe so as to be in operative fluid communication with an inlet of the probe, wherein the fluid supply console includes a conduit which is arranged, in use, to provide a thermal fluid to the probe to enable heating or cooling by the probe, and wherein the fluid supply console includes a heating element which is configured to selectively heat the thermal fluid before it is provided via the conduit to the inlet of the probe so as to enable selective heating of the probe in use.

In accordance with another aspect of the present disclosure there is provided a method of applying cryoablation treatment, the method comprising:
providing a probe which has a hollow body that defines an inlet and an outlet, the inlet configured to receive a thermal fluid for circulation through the hollow body, and the outlet configured to discharge the thermal fluid from the hollow body;
providing a fluid supply console remotely and in fluid communication with the inlet of the probe;
by the fluid supply console, operatively supplying the thermal fluid to the probe so as to enable heating or cooling by the probe; and
by a heating element of the fluid supply console, selectively heating the thermal fluid before it is provided to the inlet of the probe so as to selectively heat the probe in use.

Embodiments of the present disclosure will now be described, by way of example only, with reference to the accompanying drawings.

### BRIEF DESCRIPTION OF THE DRAWINGS

In the drawings:

FIG. 1 is a front view of an exemplary embodiment of a fluid supply console for a cryoablation system according to aspects of the present disclosure;

FIG. 2 is a rendering that shows a three-dimensional view of the console of FIG. 1;

FIG. 3 is a diagrammatic representation of an exemplary embodiment of a distal tip of a probe or cryoprobe that can be used together with the console of FIG. 1;

FIG. 4 is a diagrammatic representation of distal tip and distal portion of an exemplary probe illustrating more detail of an inner tube or fluid conveying tube, also showing an exemplary diagrammatic sectional view taken along line A-A in this Figure;

FIG. 5 is an exemplary embodiment of a probe or cryoprobe that can be used in an exemplary cryoablation system of the present disclosure;

FIG. 6 is a high-level block diagram of an exemplary embodiment of a cryoablation system;

FIG. 7 is a high-level block diagram of another exemplary embodiment of a cryoablation system;

FIG. 8 is a front view of an exemplary heating element or heater that may form part of the fluid supply console, or that may be integrated in the fluid supply console;

FIG. 9 is a right-side view of the heater;

FIG. 10 is a sectional view taken along line A-A in FIG. 9, illustrating internal components of the heater in more detail;

FIG. 11 is a three-dimensional view of the heater, a three-dimensional rendering of the heater, and a three-dimensional rendering showing a sectional view through the heater;

FIG. 12 is a diagram illustrating stresses in the heater of FIGS. 8-11 during exemplary testing or test simulations;

FIG. 13 shows photographs of an exemplary embodiment of the heater being partially disassembled and assembled;

FIG. 14 shows photographs of further exemplary prototypes of a heater for use in a cryoablation system according to aspects of the present disclosure;

FIGS. 15-16 are renderings of exemplary embodiments of internal components of a fluid supply console for a cryoablation system illustrating an exemplary manifold and valve system that may be implemented;

FIGS. 17-18 are diagrammatic renderings of a top view and a three-dimensional view of an exemplary embodiment of a fluid supply console for a cryoablation system, also showing the internal components of the console;

FIGS. 19-20 are three-dimensional renderings of an exemplary embodiment of a heater for use in a cryoablation system showing exemplary testing or test simulations of the heater;

FIG. 21 is a front view of an exemplary probe according to an embodiment of the present disclosure;

FIG. 22 is a sectional view taken along line B-B in FIG. 21;

FIG. 23 is an enlarged view of a portion of the probe designated “D” in FIG. 22;

FIG. 24 is a front view of another exemplary probe according to an embodiment of the present disclosure;

FIG. 25 is a sectional view taken along line C-C in FIG. 24;

FIG. 26 is an enlarged view of a portion of the probe designated “E” in FIG. 25;

FIG. 27 is a high-level block diagram of pneumatic architecture and/or fluid flow architecture of an exemplary embodiment of a cryoablation system;

FIGS. 28-29 are high-level block diagrams of exemplary embodiments of a cryoablation system;

FIG. 30 is another high-level block diagram of an exemplary embodiment of a cryoablation system;

FIG. 31 is a high-level block diagram of pneumatic architecture and/or fluid flow architecture of an exemplary embodiment of a cryoablation system;

FIG. 32 is a flow diagram illustrating an exemplary method of applying cryoablation treatment;

FIG. 33 is a high-level block diagram of exemplary control software architecture that may be implemented by an exemplary embodiment of a cryoablation system of the present disclosure;

FIGS. 34-39 are three-dimensional cutaway views of further exemplary embodiments of heaters or heating elements according to aspects of the present disclosure;

FIG. 40 is a sectional view of an exemplary heating element illustrating exemplary flow of thermal fluid therethrough;

FIG. 41 is a three-dimensional view of an exemplary fluid connector for connecting the probe to the fluid supply console, also showing enlarged views of an exemplary button and locking device;

FIG. 42 is an exemplary sectional view of the fluid connector of FIG. 41 when it is unplugged;

FIG. 43 is an exemplary view of a console with the fluid connector of FIG. 41;

FIG. 44 is an exemplary sectional view of the fluid connector of FIG. 41 when it is plugged in;

FIG. 45 is a three-dimensional view of another exemplary fluid connector for connecting the probe to the fluid supply console, also showing enlarged views of an exemplary lever and locking device;

FIG. 46 is an exemplary sectional view of the fluid connector of FIG. 45 when it is unplugged;

FIG. 47 is an exemplary sectional view of the fluid connector of FIG. 45 when it is plugged in;

FIG. 48 is a diagrammatic sectional view of an exemplary probe tip showing flow of thermal fluid therethrough;

FIGS. 49-51 are three dimensional views of exemplary probe tip configurations according to aspects of the present disclosure;

FIG. 52 is an exemplary sectional view through an exemplary probe tip, showing flow of thermal fluid therethrough;

FIGS. 53-58 are exemplary sectional views through exemplary probe tips, showing further exemplary probe tip configurations according to aspects of the present disclosure;

FIG. 59 is a side view of another exemplary embodiment of a probe according to aspects of the present disclosure;

FIG. 60 is an exemplary sectional view through the probe tip of FIG. 59 taken along line D-D;

FIG. 61 is an exemplary sectional view through the probe of FIG. 59 taken along line E-E;

FIG. 62 is an exemplary three-dimensional cutaway view of the probe of FIG. 59; FIG. 63 is a high-level block diagram illustrating exemplary components of a cryoablation system according to aspects of the present disclosure;

FIG. 64 is an exemplary state machine showing more detail of a state machine that may form part of the system of FIG. 63;

FIG. 65 is a high-level block diagram illustrating further exemplary components of the cryoablation system of FIG. 63;

FIG. 66 is another high-level block diagram illustrating yet further exemplary components of the cryoablation system of FIG. 63; and

FIG. 67 is a high-level block diagram of an exemplary pneumatic architecture and/or fluid flow architecture of an exemplary embodiment of a cryoablation system in which a heating elements is implemented for each of a number of heating channels for cryoprobes.

### DETAILED DESCRIPTION WITH REFERENCE TO THE DRAWINGS

Embodiments of the present disclosure relate to systems, methods and apparatuses for cryoablation treatment, or cryo-surgical treatment. A probe or needle may be used that includes an elongate hollow body, also termed a needle body. The hollow body may in certain embodiments include an inner tube or passage for thermal fluid. The probe or needle body may extend from a proximal end toward a distal end thereof, with the distal end being closed, but preferably sharpened so as to be inserted into a patient, or mammal. It will be appreciated that animals or other mammals (in addition to humans) may also be treated by the systems and methods of the present disclosure. The probe may be termed a cryoprobe. It will be appreciated that the probe may be arranged to provide cooling, e.g., by cryoablation freezing during which biological material, water, or tissue surrounding the probe is cooled significantly and/or frozen. It will further be appreciated that the probe may also be arranged to provide heating, e.g., by “thawing” the cryoprobe after it was cooled or frozen, i.e., so as to heat the probe body in order to thaw or heat biological material or tissue that surrounds it. The probe or cryo-needle may also be termed an incision device or a piercing rod with one or more internal voids for circulation of thermal fluid. Thermal fluid such as air, argon or other gas(es) or fluids may be circulated inside the needle body in either direction, e.g. from an inner region of the needle toward an outer region or the other way around.

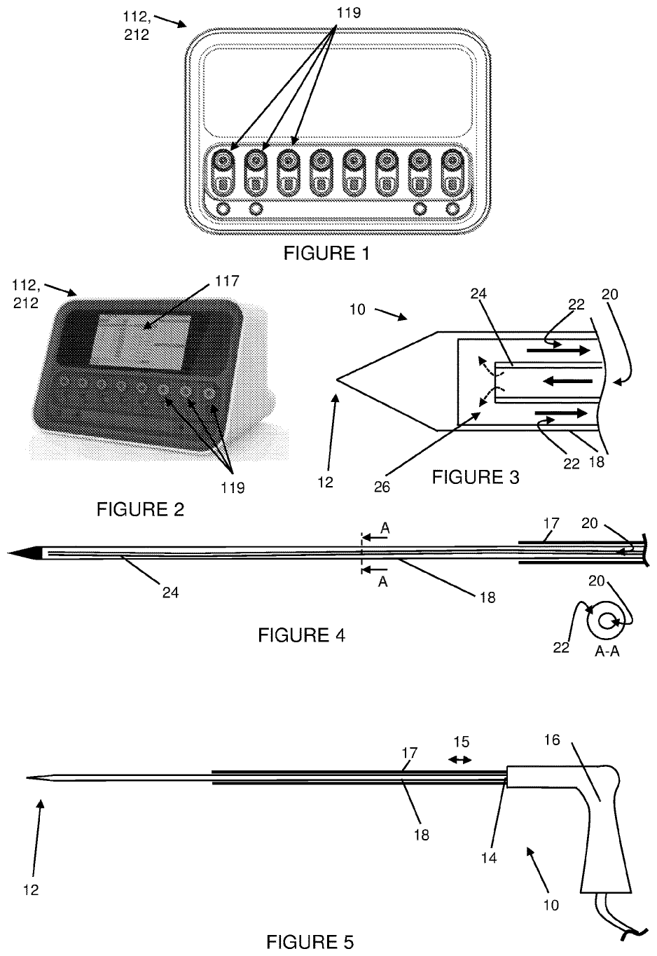

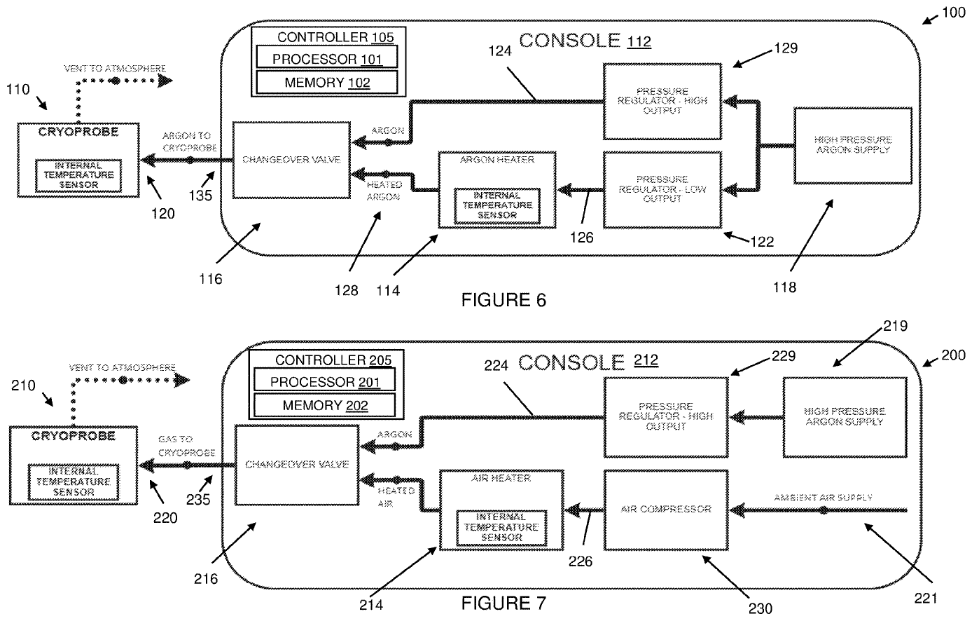

A console or central control unit/module may be provided to supply thermal fluid to the probe or needle, and connecting tubing, valves and ports may be provided to provide a fluid interface between the console and the probe or pointed needle device. The console may be in fluid communication with a fluid intake or inlet of the probe, and the console may be arranged to provide the thermally conductive fluid to the probe, so as to heat or cool the probe in use. The probe, or parts of the probe needle may be frozen in a first cooling step or cooling mode of the console/system (and/or biological material, tissue, or water that surrounds it may be frozen), and it may be thawed in a second heating step of the console/system, e.g., heating the probe body in order to thaw or heat biological material or tissue that surrounds it. Freezing or cooling may be implemented by way of the Joule-Thomson effect, or by way of heat transfer through convection and conduction. Heating may also be provided by way of heat transfer through convection or conduction. A plurality of pressure regulating devices may form part of the fluid supply device or console of the system. Optionally, pressure regulating device(s) may also form part of the probe. Embodiments of the present disclosure relate to a cryoablation system, a fluid supply console (also referred to as a fluid supply module or unit), and a probe (which may also be termed a cryoprobe) for use in the cryoablation system. An exemplary embodiment of a fluid supply console is depicted in FIGS. 1 and 2, and an exemplary embodiment of the probe or cryoprobe is depicted in FIGS. 3 to 5. Exemplary embodiments of the cryoablation system are also shown in the block diagrams of FIGS. 6 and 7. Further features of the cryoablation system will be described in more detail below. Throughout the Figures, similar reference numerals may be used to designate similar features or components.

Referring to FIG. 6, in one embodiment, a cryoablation system (100) may include a probe (110) or cryoprobe. An exemplary embodiment of a probe (10) that may be used in the cryoablation system of the present disclosure is shown in FIG. 5. Optionally, a sliding shaft (17) or sleeve may be provided, however, it will be appreciated that the present disclosure is not limited to the use of a sliding shaft or insulating sleeve. The probe (10) may be used on its own together with the various systems of the present disclosure, or another type of probe may be used. The probe (10) may have a distal end (12) or tip which may be closed and sharpened, so as to facilitate it to be inserted into a patient (not shown) for cryo-treatment or cryoablation treatment (e.g., to destroy malignant tissue by cold or freezing temperatures). The probe may be elongate and hollow, and it may also have a proximal end (14) or a proximal portion. Optionally, a handle (16) may be provided to facilitate insertion into the patient in use, and manoeuvring of the probe (the probe may also be termed a cryo-needle).

Referring to FIGS. 3 to 5, the probe (10) may have a hollow body (18) that defines an inlet (20) and an outlet (22). The inlet (20) of the probe may, in use, be connected to a fluid supply console (112) (e.g., as shown in FIG. 6). The console (112) will be described in more detail below. The inlet (20) may, for example, be defined by the hollow body (18) of the probe, e.g., by way of an inner tube (24) which extends inside the hollow body (18) of the probe. The inner tube (24) can be seen in FIG. 4, and in the diagrammatic representation of the distal tip of the probe shown in FIG. 3. Many other configurations are of course possible.

The inlet (20) of the probe (10) may be configured to receive thermal fluid for circulation through the hollow body (18) of the probe (10), e.g., as is designated by the directional arrow pointing left inside the inner tube (24) in FIG. 3. The outlet (22) of the probe (10) may be configured to discharge the thermal fluid from the hollow body (18). The outlet (22) may also be termed a vent, and it may be arranged to vent the thermal fluid (e.g., back to atmospheric pressure) away from the distal tip (12) of the probe (10). Embodiments of the present disclosure may enable the thermal fluid to expand, preferably rapidly, after entering the hollow probe body (18). In the exemplary embodiment, the thermal fluid may enter the inlet (20), move through the inner tube (24) and expand into the hollow body (18) at a distal end region (26) of the inner tube (24). This may cause the thermal fluid to reduce its temperature, which may, in turn, cause cryo-cooling or cryoablation, so that the probe body (18) may freeze, or so that its temperature is substantially lowered, so as to cause cryoablation treatment of the surrounding tissue (e.g., when inserted into a patient).

The probe (10) may also be termed a cryoprobe, and a plurality of cryoprobes or probes may be implemented in the systems and methods of the present disclosure. The cryoprobe may include a needle portion that has an outer tube having a distal section or distal portion with a gas supply line (also termed an inner tube (24)) positioned coaxially within the outer tube or probe body (18). The gas supply line (24) may be arranged to supply pressurized argon gas that expands into an expansion chamber located near the distal portion (12) of the probe body (18), such that the central gas supply line (24) terminates in the expansion chamber. This expansion of the pressurized argon gas is what may cause the rapid cooling of the gas according to the Joule-Thomson effect. The cooled gas absorbs heat from the metallic walls of the cryoprobe by convection and conduction which causes water to form an ice-ball on the outer surface of the cryoprobe needle (e.g., inside a patient's body in use). The depressurized gas may then be vented back out of the cryoprobe. Other than the needle, the cryoprobe may include the handle (16) for the physician to hold and manoeuvre the needle, and a tube (for thermal fluid) connected to the console. To control the length of the ice-ball that forms on the cryoprobe needle, a sleeve (17) can be implemented (see FIG. 4) that slides over the needle, providing an insulating layer between the cooled outer metal surface of the needle and the patient. The length of the sleeve can be adjustable (15) by the user to create ice-balls of different lengths. The cryoprobe(s) may have a temperature sensing element inside the needle or probe body (18) for measuring the expanded gas temperature which can be displayed on the console graphical user interface. The cryoprobes can also have a digitally controlled colour element like an RGB LED embedded in the handle which will correlate with the probes displayed on a console display (117).

An exemplary embodiment of a fluid supply console (112) and its housing or enclosure is shown in FIGS. 1 and 2. It will be appreciated that the housing may also be used in conjunction with other embodiments of the present disclosure, for example the embodiment of the console (212) shown in FIG. 7 (described in more detail elsewhere in the present disclosure). In embodiments of the present disclosure, thermal fluid may be supplied to the inlet (20) of the probe (10), e.g., by the fluid supply console (112, 212).

The fluid supply console (112, 212) may be remote from the probe (10) and in fluid communication with the inlet (20) of the probe (10). Piping, or tubing (not shown) may be provided to couple the probe fluidically to the fluid supply console or unit. The probe (10) may be handheld (e.g., as shown in FIG. 5), however embodiments may also be possible in which the probe is mechanically or electrically operated (optionally automatically or in a controlled manner), e.g., robotically, or by way of a moving apparatus or machine. The probe may be fairly simple to manufacture, and it may include the hollow body (e.g., made from surgical steel or another metal, rigid material or composite). The inlet may simply be connectable to a fluid supply conduit or pipe that connects it to the separate fluid supply console, which can be remotely powered and remotely located from the probe. This may be advantageous, as the probe's construction can be kept simple and cost effective, while more complex components can be provided separately in the console.

Referring to FIG. 6, the fluid supply console (112) may be arranged, in use, to provide the thermal fluid to the probe (10) to enable heating or cooling by the probe. It will be appreciated that other embodiments of the fluid supply console of the present disclosure may be arranged in a similar fashion. In the present embodiment, the fluid supply console (112) includes a heating element (114) which is configured to selectively heat the thermal fluid before it is provided to the inlet (20) of the probe (10), so as to enable selective heating of the probe (10) in use. This may be advantageous, because once the probe is substantially cooled or frozen, it may become necessary for a user (e.g., a surgeon, doctor or their assistant) to remove the probe. In practice, this may often need to be done quickly. The present disclosure may enable thawing or heating of the probe, by selectively supplying heated thermal fluid to the probe, e.g., after the thermal fluid is heated by the heating element (114). This may be advantageous, since rapid thawing or rapid heating of the probe body (18) may be implemented so as to speed up the removal process of the probe. The heating element (114) may also have its energy source provided at the console, which may enable more energy to be provided to the thermal fluid by the heating element. Electrical energy or other thermal energy such as combustion energy in the case of a gas operated heating element may be provided at the console, remotely or separately from the probe. Still referring to FIG. 6, the fluid supply console (112) of the present embodiment may include a valve system and/or a valve (116). The valve may for example be a changeover valve. The valve may be operable and/or selectable between a first state and a second state. In the first state the valve (116) may be configured to provide the thermal fluid to the probe (10, 110), e.g., for cooling. In the second state, the valve may be configured to provide the thermal fluid which is heated by the heating element (114) to the probe (10, 110). In the present embodiment of the fluid supply console (112), a single type of thermal fluid may be used, presently argon. A pressurized argon supply (118) (presently in the form of a pressurized argon tank, reservoir or the like) may be included in the console, or it may be fluidically coupled to the console. One or more pressure regulators may be provided. It will be appreciated that the embodiment described with reference to FIG. 6 may include or implement one or more features of the other embodiments of the present disclosure and vice versa.

In the present embodiment, a first pressure regulator (129) is provided, e.g., for a first (preferably higher-pressure) argon flow stream (124); and a second pressure regulator (122) is provided, e.g., for a second (preferably low-pressure) argon flow stream (126). In other words, the first pressure regulator may provide a high-pressure fluid output, and the second pressure regulator may provide a low-pressure fluid output (i.e., lower than that of the first pressure regulator). Both argon flow streams, or fluid flow pathways may be supplied by a single argon supply (118) or a single pressurized thermal fluid supply, however a plurality of fluid supplies or argon supplies may also be used if necessary. It may, however, be advantageous to use a single fluid supply, such as a single argon tank, and splitting up the flow as illustrated in FIG. 6.

The second fluid pathway or fluid flow stream may, optionally, be heated by the heater or heating element (114), e.g., when the console (112) receives a user input. The heater may be mechanically or electrically actuated, preferably automatically, once the user input is received (e.g., digitally). Hence, heated thermal fluid (e.g., heated argon (128)) may be provided by the heater or heating element (114). Optionally, an internal temperature sensor may be provided at or near the heating element, e.g., to provide temperature feedback to a processor which may be associated with the console.

The fluid supply console (112, 212) of the present disclosure may include one or more processor(s) (101, 201), and a memory (102, 202) that may optionally form part of a controller (105, 205). The controller may, for example be housed by the console which may also include a user interface or display. The present disclosure extends to embodiments of the system (100, 200) in which the console (112, 212) includes the processor (101, 201) for executing the functions of components described in the present disclosure, preferably automatically or upon receiving user input. The components of the console (112, 212) may be provided by hardware or by software units executing on the processor(s) (101, 201) associated with the respective console (112, 212) and/or controller(s) (105, 205). The software units may be stored in the memory component (102, 202) and instructions may be provided to the processor (101, 201) to carry out the functionality of the described components. In some cases, for example in a cloud computing implementation, software units arranged to manage and/or process data on behalf of the console (112, 212) may be provided remotely. Some or all of the components may be provided by a software application downloadable onto and executable on the console (112, 212) or fluid supply console. It will be appreciated that the embodiments described with reference to FIGS. 6-7 may include or implement one or more features of the other embodiments of the present disclosure and vice versa.

The changeover valve (or any of the other valve(s), pressure regulator(s), compressor(s) or the heater(s)) may be automatically operated or actuated, e.g., by receiving data or instructions from the processor (101, 201). For example, when cooling or cryoablation of the cryoprobe or probe is required, the user may select a cooling option, and the changeover valve may be actuated into the first state to provide the thermal fluid (e.g. Argon) to the probe, e.g., for cooling by way of the Joule-Thomson effect, or by virtue of the thermal fluid itself being cold (e.g. liquid nitrogen or another liquid or cooling fluid). Cooling (in other words, heat transfer from the tissue/matter surrounding the probe's tip and hollow body) may be allowed to proceed and the tissue may be frozen, e.g., during Cryo-treatment. Once the surgeon, doctor, or assistant or another user requires heating, they may press a button (not shown) or provide other input data to the processor (101, 201), after which the changeover valve may be actuated to its second state so as to provide the thermal fluid which is heated by the heating element (114) to the probe. These actuations of the changeover valve may be performed, e.g., by receiving actuation data or instructions digitally or by analogue means from the processor. Embodiments are also possible in which pneumatics or hydraulics are used to implement control of the various components of the console (112, 212). In the embodiment of FIG. 6, Argon is used as thermal fluid for both fluid paths (124, 126) (i.e., first and second fluid paths), and heating or cooling of the probe is performed by the changeover valve or valve being actuated (e.g., by a solenoid valve or the like) between the first state and the second state of the valve (i.e., by selecting the first fluid path or the second fluid path). Argon or other thermal fluid may then be supplied to the probe (e.g., to the inlet (20) via a conduit, pipe or fluid passage connected to the console (112, 212)), and it may flow through the hollow body (18) of the probe (e.g., as is diagrammatically illustrated in FIG. 3). After cooling (or heating) has been performed in the probe body, the thermal fluid may be vented or returned through the outlet (22), e.g., to atmospheric pressure. Depending on the particular arrangement, the thermal fluid may be vented to ambient (e.g., into the room or surroundings of the probe's handle), or the thermal fluid may be returned via a conduit back to the console (112, 212).

The fluid supply console (112) may be operable to automatically select the first state and/or the second state of the valve (116) and/or valve system so as to selectively provide heating or cooling of the probe (10, 110) in use, for example upon receiving input from a user which is indicative of heating or cooling being required by the probe. The fluid supply console (112) may be arranged to provide a single type of fluid as thermal fluid (e.g., argon) to the probe (10, 110) to facilitate cooling by the probe in use, for example by cryoablation cooling and/or by freezing the probe, and the single type of fluid (e.g., argon) may be heated by the heating element to facilitate thawing or heating of the probe body, or so as to heat the probe body in order to thaw or heat biological material or tissue that surrounds it. The heating element (114) may be arranged to selectively heat the single type of thermal fluid before it is provided to the probe (10, 110), so as to selectively heat or thaw the probe body. Similar features may be implemented by the console (212) of FIG. 7. It will be appreciated that the present embodiment may include or implement one or more features of the other embodiments of the present disclosure and vice versa.

An alternative embodiment of the console (212) is shown in FIG. 7. This embodiment is similar to the embodiment described with reference to FIG. 6, however it includes two different types of thermal fluid, presently argon (219) (e.g., provided by a pressurized argon supply/container) and air (221) (e.g., provided from the atmosphere, or a pressurized air container). It will be appreciated that any number of fluid types may be used, depending on practical considerations. The fluid supply console (212) may be arranged to selectively provide one or more thermal fluids (219, 221) to the probe. The fluid supply console (212) may be arranged to selectively provide a first thermal fluid (219) (e.g., argon) for cooling the probe (10, 210), and a second thermal fluid (221) (e.g., air) for heating the probe (10, 210). Optionally, a compressor (or a plurality of compressors) may be provided to pressurise one or more of the thermal fluids. In the present embodiment, an air compressor (230) is connected to an ambient air supply (221). Similar to the embodiment of FIG. 6, a first fluid flow path (224) may be provided for a first thermal fluid (e.g., for cooling of the probe by Argon), and a second fluid flow path (226) may be provided for a second thermal fluid (e.g. for air which can be used to heat the probe). A heating element or heater (214) may be provided for either one of the first and second fluid flow paths, presently for the air flow path. The heating element (214) may also have an internal temperature sensor, for example to provide temperature feedback to the processor (201) where it may be analysed and appropriate actions may be performed, (e.g., increasing or decreasing heating of the relevant thermal fluid by the heater). A pressure regulator (229) may be provided similarly to that shown in FIG. 6. It will be appreciated that the present embodiment may include or implement one or more features of the other embodiments of the present disclosure and vice versa. A pressure regulator (220) may also be provided similarly to the other embodiment of FIG. 6, e.g., to regulate the argon pressure before it is directed by the changeover valve to the probe (preferably under control of the processor).

The heating element (214) (presently an air heater) of the fluid supply console (212) may be arranged to selectively heat the second thermal fluid (221) (presently air) before it is provided to the inlet (20) of the probe (10, 210), for example to thaw, heat, or “defrost” the probe, or so as to heat the probe body in order to thaw or heat biological material or tissue that surrounds it. The heating element may be controlled by the processor or controller (205). The heating of the probe may be performed by convection or conduction heat transfer of the heated fluid to the probe body (18), e.g., via a conduit connecting the console (212) and the inlet of the probe body (18).

In embodiments of the systems (100, 200) of the present disclosure, the fluid supply console (112, 212) may include a valve or valve system (116, 216) which may be arranged to selectively provide the first thermal fluid (124, 224) for operatively cooling the probe (110, 210, 10) in use. Alternatively, or in addition, the valve or valve system (116, 216) may be arranged to selectively provide a second thermal fluid (126, 226) for operatively heating the probe. The first and second fluids may be the same (e.g., both may be argon), or the first and second fluids may be different (e.g., argon for cooling and air for heating). The fluid supply console (110, 210) may include the valve or valve system which may be arranged to selectively provide the first thermal fluid for operatively cooling the probe, and/or the valve or valve system may be arranged to selectively provide the second thermal fluid for operatively heating the probe in use.

The first thermal fluid may be any fluid selected, so as to provide cooling by way of the Joule-Thomson effect, or in another way. For example, in an exemplary embodiment, the first thermal fluid may be argon, which is rapidly expanded when leaving the inner tube (24) of the probe (10), thus causing its temperature to drop significantly and freezing or significantly cooling tissue or material adjacent to the probe (especially near its distal tip or distal end (12)). However, it will be appreciated that many other configurations of the probe body are possible, and embodiments may be possible in which fluid is circulated for cooling (or heating) in another way (e.g. by internal conduits inside the body of the probe itself, e.g., without using an inner tube). The entire length of the probe body may be cooled by the thermal fluid, whether it be due to the Joule-Thomson effect, or due to the fluid itself being cold (e.g., to facilitate heat transfer by conduction and convection). For example, it is also envisaged that liquid nitrogen or another gas, liquid or fluid can be used instead of argon, even though it is not presently preferred. Similarly, other gases, liquids or fluids may be used for heating, instead of argon or air (e.g., helium or perhaps even heated water). The thermal fluid used for heating may also facilitate heat transfer to the probe body, e.g., by convection or conduction. In other words, the second thermal fluid (126, 226) may be any fluid selected to provide heating by way of thermal conduction or thermal convection. The first fluid and/or the second fluid may be selected so as to be thermally conductive. The thermal fluid or thermal fluids may be selected from a list including: argon or argon based fluids, air, helium or helium based fluids, nitrogen or nitrogen based fluids, liquid nitrogen, inert or ideal gases, or any other gas or liquid capable of thermally heating or cooling the probe, as the case may be.

The fluid supply console (112, 212) may be connected to a pressurized thermal fluid supply (e.g., 118, 219, 221, 230), and/or it may be connected to one or more pressurized thermal fluid supplies or supply reservoir(s). For example, a first reservoir for pressurized argon (118) may be provided and in fluid communication with the fluid supply console (112). Additionally, or alternatively, a second reservoir for pressurized air (not shown) may be provided and in fluid communication with the fluid supply console (212). The fluid supply console (112, 212) may be used in conjunction with a cryoablation system according to the present disclosure. However, it will be appreciated that the fluid supply console may be manufactured separately and/or sold separately from the cryoprobe or probe. This may be advantageous, since a single fluid supply console may be arranged to be modular, and/or it may be arranged to be used together with a plurality of probes or cryoprobes. For example a fluid supply console may be provided with a plurality of fluid supply channels, each being similar to that of FIG. 6, or FIG. 7, as the case may be. This may enable a plurality of probes to be connected to a central fluid supply console or hub, so as to enable each of the plurality of probes to be controlled separately for heating and cooling of the respective probe in use.

Referring again to FIGS. 3 to 5, each probe (10) may have the hollow body (18) which extends from a proximal end (14) of the hollow body to a distal end (12) thereof. The probe may include a fluid conveying tube (24) extending inside the hollow body from the proximal end toward the distal end. The fluid conveying tube or inner tube may be arranged to open into the hollow body (18). The distal end (12) of the hollow body may be closed so that thermal fluid is enabled to enter the hollow body through the fluid conveying tube and to heat or cool the body of the probe, as the case may be. The distal end of the probe body may be sharpened, or otherwise arranged or configured to be inserted into a patient, e.g., for cryoablation treatment or cryo-treatment.

The present disclosure extends to a cryoablation system (100, 200) comprising: a fluid supply console (112, 212) which may be remotely connectable to the probe (10, 110, 210) so as to be in operative fluid communication with an inlet (120, 220) of the probe (10, 110, 210). The fluid supply console (112, 212) may include a conduit (135, 235), or one or more conduits which may be arranged, in use, to provide a thermal fluid to the probe to enable heating or cooling by the probe. The fluid supply console (112, 212) may include the heating element or heater (114, 214) which may be configured to selectively heat the thermal fluid before it is provided via the conduit to the inlet of the probe so as to enable selective heating of the probe in use. It will be appreciated that the present embodiment may include or implement one or more features of the other embodiments of the present disclosure and vice versa.

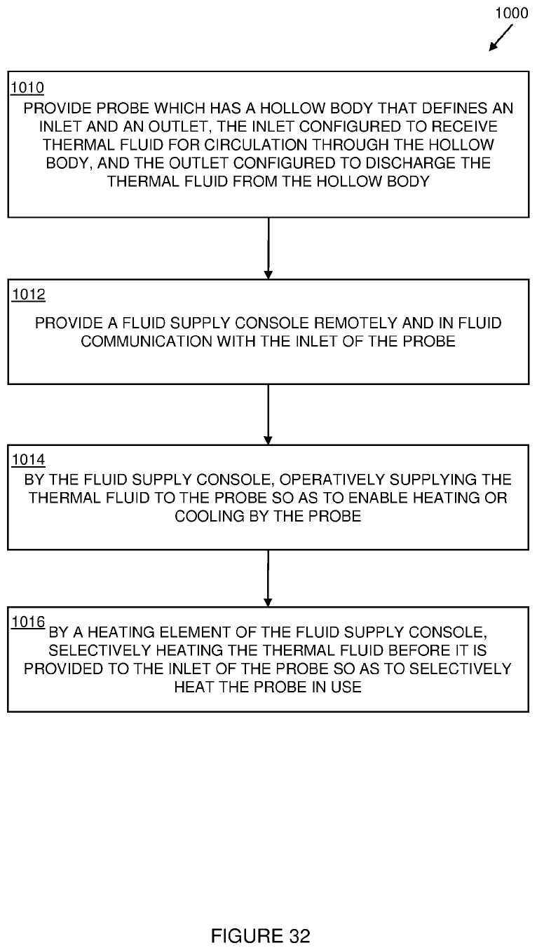

Referring to FIG. 32, the present disclosure extends to a method (1000) of applying cryoablation treatment. The method may include providing (1010) a probe (10, 110, 210) which may have a hollow body (18) that defines an inlet and an outlet. The inlet may be configured to receive thermal fluid for circulation through the hollow body. The outlet may be configured to discharge the thermal fluid from the hollow body. The method may further include providing (1012) a fluid supply console (112, 212) remotely and in fluid communication with the inlet (20) of the probe (10, 110, 210). The method may further include, by the fluid supply console (112, 212), operatively supplying (1014) the thermal fluid (118, 219, 221) to the probe (10, 110, 210) so as to enable heating or cooling by the probe in use. The method may yet further include, by a heating element (114, 214) of the fluid supply console (112, 212), selectively heating (1016) the thermal fluid before it is provided to the inlet of the probe (10, 110, 210) so as to selectively heat the probe in use. It will be appreciated that the present embodiment may include or implement one or more features of the other embodiments of the present disclosure and vice versa.

The cryoablation system of the present disclosure may, for example be used for cryoablative destruction of tissue during minimally invasive procedures. The cryoablation system may be used as a cryosurgical tool in the fields of general surgery, dermatology, neurology (including cryoanalgesia), thoracic surgery (with the exception of cardiac tissue), ear nose and throat (ENT), gynecology, oncology, proctology, and urology. This system may be used to destroy tissue (including prostate and kidney tissue, liver metastases, tumours, and skin lesions) by the application of substantially or extremely cold temperatures.

The cryoablation system of the present disclosure may include a pressurized gas source, which may be referred to as the console gas source (or a gas source associated with the console, or forming part thereof), and one or more cryoprobes connected to the console. The console may be used together with a gas or fluid reservoir, cylinder or tank, or the reservoir may be integrated into the console. Alternatively, or in addition, the console may include or be connected to a compressor, e.g., for compressing fluid or gas that is to be used in the systems and methods of the present disclosure. E.g., an air compressor may be used, or another type of fluid compressor. The console can also accommodate one or more temperature probes, temperature sensors to assist with monitoring the extent of the cooling caused by the cryoprobes inside the patient. Temperature data may be received by the console from the temperature sensor at or near the probe(s). Alternatively, or in addition, separate temperature sensing elements or temperature sensing probes may also be connected to the console and inserted into the patient (e.g., near the cryo-probe), e.g., to feed temperature data or a temperature indicating signal to the console controller. The temperature probe(s) may be shaped similarly to the cryo-probes or hollow probes that are used for cooling or heating (i.e., to form the ice-ball and/or to thaw it).

Each cryoprobe may have a temperature sensor that provides feedback to the console and user (e.g., via the display). Separate ports may be available for additional, dedicated temperature probes that are inserted into the patient to provide feedback of temperatures in the surrounding tissue. This may provide feedback e.g., which may be indicative of the ice-ball's size. These temperatures may also be displayed by the console to assist the user during the procedure.

It is further envisaged that embodiments may also be possible in which one or more temperature sensors may be provided in or along the probe (i.e., the cryo-probe) so as to sense temperature data from various points along the probe body in near real-time. This may enable a doctor or user to review temperature data in real-time on a display associated with the console or this data may be automatically analysed by the console so as to enable actions to be taken, e.g., preventative or emergency actions to be performed. The console may be considered to be reusable. In presently preferred embodiments, the cryoprobes or probes are single-use disposable items. This may be advantageous, since this could inhibit or prevent the spread of infection, and it can protect both doctors and patients. The more complex or expensive components of the system may be provided in the console which may be reusable, so as to be more cost effective, especially in countries with limited resources when it comes to medical equipment. It will be appreciated that these embodiments may include or implement one or more features of the other embodiments of the present disclosure and vice versa.

As shown in the exemplary embodiment in FIGS. 1 to 2, the console, which may form part of the systems and methods of the present disclosure, may be a portable console with a touch screen interface that may allow a user to control the application of pressurized argon gas (or other thermal fluid) to one or more cryoprobes. The console may alternatively be stationary, or non-portable in certain embodiments. The console may be separate from the probe(s).

The console may use the Argon gas source (e.g., 118 in FIG. 6) which may be provided by an Argon gas tank. The console may be arranged to regulate the incoming gas pressure to the appropriate pressures for cryoablative procedures to be performed. The console may have multiple channels or fluid paths for connecting a plurality of cryoprobes. The console (112, 212) may be enabled to independently control gas flow to each channel. The system may both freeze and actively thaw/heat the cryoprobe needles or probe body(ies). The freeze functionality may be implemented by applying high pressure argon gas to the cryoprobe. Active thawing/heating may be implemented by applying low pressure, electrically heated Argon gas to the cryoprobe. It is envisaged that the heating element (114) may be an electric heating element arranged for heating argon gas, or another type of thermal fluid.

In the exemplary embodiment of FIG. 6, the pneumatic path (or fluid flow path(s)) of the console may include the following features. Two pressure regulators (122, 129) may be provided, one for high pressure argon used for cryoprobe needle freezing, and one for low pressure heated argon gas used for active thawing/heating. A manifold may be provided, and one or more solenoid valves may be implemented for controlling the flow path of the argon. Similarly, one or more solenoid valves may be provided to control the fluid flow path of argon or air in the embodiment shown in FIG. 7. The solenoid valve(s) may be controlled by the controller (105, 205) to automatically control flow of the first thermal fluid and/or the second thermal fluid.

It will be appreciated that the fluid flow path(s) shown in FIG. 6 is merely one exemplary embodiment (100) of the pneumatic or fluid flow-path where argon gas is used for both heating and cooling of the cryoprobe needle or probe body. The second exemplary embodiment (200) of the pneumatic path is shown in FIG. 7 where compressed and heated ambient air may be used for heating the cryoprobe needle instead of argon gas. Both embodiments may implement active gas heating.

One of the benefits of implementing active heating inside the console is that it may reduce the complexity of the cryoprobe compared to other arrangements, e.g., if the heating was to be implemented in the needles or in a handle of the needle itself. This may substantially reduce the cost of the needles. Another major benefit may be that of electrical safety. With no heating elements in the cryoprobe, there is no need for a high-power electrical source inside the probe needle, which makes the needle(s) inherently safer, and also more lightweight and thus easier to manoeuvre (not to mention the option of making the needles disposable as mentioned elsewhere in the present disclosure, along with the advantages of that). This may also provide advantages in ease of manufacturability, reduced failure or resilience against failure, reduced component counts, etc.

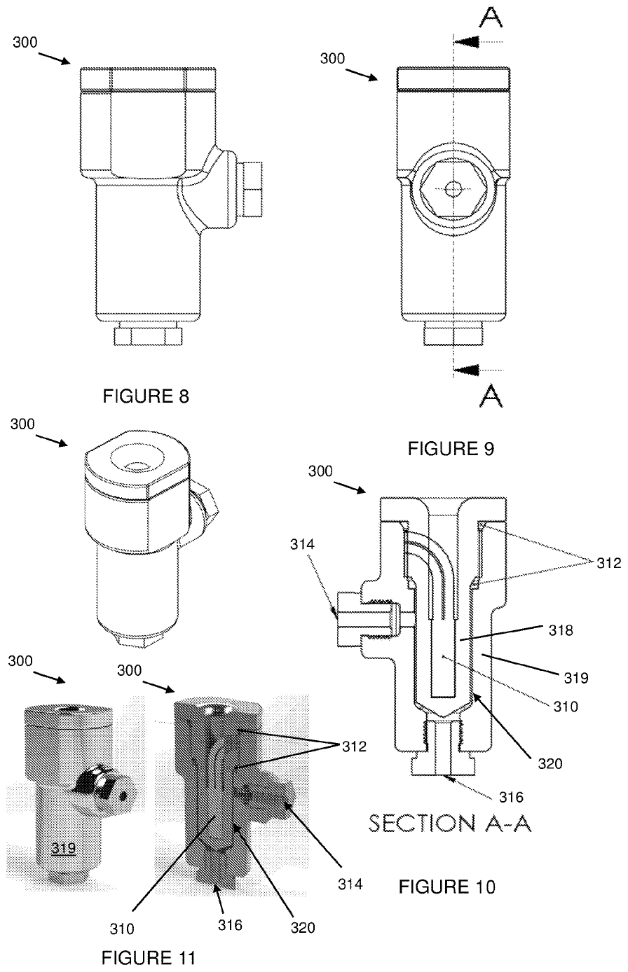

Turning now to FIGS. 8-13, there is shown an exemplary embodiment of a heater or heating element (300). The heater or heating element may be arranged to heat thermal fluid (e.g., as is designated by the reference numerals (114, 214) in the exemplary block diagrams of FIGS. 6-7). Referring to the sectional view in FIG. 10, the exemplary heater may include a heating element (310), one or more pressure seals (312) an outlet (314) for heated fluid, and an inlet (316) for receiving non-heated or cool fluid. In the present embodiment, the heater is an electrical heater for heating the thermal fluid (whether it be air, argon or another thermal fluid). In other words, the heating element (310) may be electrically powered by a power supply (not shown) which may be associated with the console (112, 212). The heater may also be referred to as a gas heater or a thermal fluid heater. It is envisaged that other heating elements or methods may be used, for example using combustion, but electric heating may be preferable. The thermal fluid flow path may be electronically, electrically, or digitally controlled via a console graphical user interface, e.g., on the device touchscreen (117) shown in FIG. 2. A plurality of ports, conduits or channels may be provided for fluid flow paths e.g., to a plurality of probes. Exemplary embodiments of these ports or conduit terminals can be seen in FIGS. 1 and 2.

The exemplary embodiment of the heater shown in FIGS. 8-13 may also be termed an active gas heater (which may be integrated inside the console). The heater may include the electrical heating element (310) inserted into a shaft (318) or tube preferably made from a rigid material such as a metal or steel. The heating element (310) may be positioned or located near a central region of the heater, which may be termed a gas chamber (320). When pressurized gas or thermal fluid is applied or provided to the heater (300), the gas may enter at one port of the heater (e.g., at inlet (316)) flowing onto the heated central rod, shaft (318) or tube which transfers heat to the gas or thermal fluid as it flows through the chamber (320) and exits at a different port of the heater (300) (e.g., out the outlet (314)). It will be appreciated that the heater shown in FIGS. 8-13 is merely an exemplary heater, and many other types of heaters or other types of fluid heating arrangements or apparatuses may be used to heat the thermal fluid before being provided to the probe. In other words, active heating element(s) may be used in the console to heat gas or fluid to heat the needle, cryo-needle, or probe. It will be appreciated that the present embodiment may include or implement one or more features of the other embodiments of the present disclosure and vice versa.

The console of the various embodiments of the present disclosure may provide a freezing mode. In the freezing mode, the console may be arranged to provide thermal fluid such as argon in a pressure range of about 200 bar (2*10\hat{ }Pascal) or about 2900 psi. The console may be arranged to continuously turn on and turn off the flow of thermal fluid, at a pressure of about 200 bar (2*10∂Pascal) or about 2900 psi, to the operator selected cryoablation probe channels via command inputs from the user interface (e.g., 117).

The console of the various embodiments of the present disclosure may also provide a thaw mode or heating mode, (may also be termed a heating mode). The console may be able to provide low pressure thermal fluid (e.g., argon gas) (e.g., from 2 bar to 100 bar or about 2 bar, about 100 bar, about 25, 50, or 75 bar or anywhere in between these pressures (these may be absolute pressures, as opposed to gauge pressures which may be about 1 bar less-i.e., from 1 bar to 99 bar), as may be required in practice). The thermal fluid may be provided at temperatures up to 150° C. to the operator selected cryoablation probe channels via command inputs from the user interface (e.g., 117) for heating, but other temperatures are also possible. The console may also be arranged to perform one or more temperature measurements. For example, the console may be able to continually measure and display temperature from the operator selected temperature probes (i.e., from a probe/cryoprobe with a temperature sensing element).

Referring to FIG. 5, it will be appreciated that the probe (10) may include or be connected to piping, tubing, or a conduit for supplying the thermal fluid from the console's port(s) (119) to the probe needle or probe body. It is further envisaged that a data or signal cable or wire or other transmission medium may be provided between the probe and the console, e.g., to provide temperature feedback from the temperature sensor of the probe to the controller at the console and/or to provide data communication. The piping and/or tubing and/or the cable may be termed an “umbilical”. One or more plugs may be provided at the probe or at the probe's handle to connect the tubing or cable. The ports (119) of the console may also be termed probe ports. The console may have the associated display (117), and an emergency button may be provided, e.g., to initiate emergency thawing or heating, when the probe requires removal. One or more warning indicators or lights or sound emitting devices may also be provided, e.g., to warn the user or operator of temperature readings from the probe that exceeds, or is less than a predetermined threshold. An emergency button may be provided, e.g., to automatically perform emergency heating or emergency cooling of the probe, as the case may be. The console may also have an on/off button or power switch, a USB port or other data communication means, e.g., to provide data communications with a remote server or computer. Optionally, the console may include a fuse box, and a vent valve to vent thermal fluid (e.g., from the probe, or from the thermal fluid supply). The console may include one or more inlets for thermal fluid, and one or more outlets for thermal fluid. The outlets may for example be at the ports (119) that can be connected to probe(s). A manual vent valve may also be provided. A pressure hose or pipe may be provided, e.g., to connect an argon tank (or other thermal fluid reservoir) to the console (112, 212). to apply a suction (e.g., to a central chamber 714 of the handle of the probe in FIG. 26) (i.e., negative pressure, or a pump may be used at the console to provide this negative pressure for sucking out fluid from the probe) on the inner tube (e.g., 24) to pull thermal fluid from the outlet (22) of the probe through the probe body (18) and toward the distal end or tip (12) of the probe (10). If the outlet (22) is open to the atmosphere, this negative pressure or pumping may draw in ambient air into the needle or probe body (18), and this may cause the probe to heat up (e.g., if it is frozen or at a temperature below ambient). Thus, “hot” air may be drawn to the tip. If the outlet (22) of the probe is connected via piping to the console, hotter thermal fluid, such as air, may be drawn from the console toward the outlet of the probe, in other words venting hotter thermal fluid in a reverse directions to the directional arrows shown in FIG. 3. This may also be termed venting out in reverse. In other words, heated thermal fluid, or thermal fluid which is at a higher temperature than that of the probe may be provided in either direction (i.e., from the outer part of the hollow body (18) toward the inner tube, or from the inner tube (24) toward the outer part of the hollow body). In some embodiments, cooling fluid may also be provided in either direction, as explained above, for example when not making use of the Joule-Thomson effect, but rather the fluid itself being cold (e.g., liquid nitrogen).

It will further be appreciated that, if suction is not available, embodiments of the present disclosure may apply hotter thermal fluid to the inner tube and rely on the hot fluid/air travelling up the inner tube on the outside of the inner tube to the tip of the probe. It is also envisaged that thermal/heat conduction of the probe body (18) (may also be termed a needle body) may be used to heat or cool it in use. E.g., when stainless steel is used to make the needle body, the system may be arranged to rely on the heat conduction of the stainless-steel needle tube toward the tip from the heated fluid entering the probe inlet (reference numerals 20, or 22 in some embodiments, e.g., FIGS. 3-5, when implementing reverse flow where the outlet acts as the inlet).

Referring again to FIGS. 8-13, the heater (300) or heating system with its heating element may be arranged to provide a reduced thermal mass which may be advantageous and efficient. The volume of the chamber (320) may be relatively small, due to the close/tight tolerances between the heating element and rod (318) and an outer body (319) of the heater (300). The chamber (320) may be a peripheral chamber (preferably a narrow peripheral chamber) formed between the inner heating element (310) and the outer body (319) of the heater, so as to provide a reduced volume therein, which may reduce stagnation. The heater may also have a high-pressure rating, and the pressure seals (312) may be arranged to withstand high pressures and heat. For safety, a full system pressure at 300 bar/6000 psi (absolute pressure) may be implemented. An intended pressure and temperature range for thermal fluid of 2 to 100 bar (absolute pressure) (or from 29 to 1450 psi) and 30° C. to 150° C. may be implemented, but embodiments are possible in which thermal fluid of more than 150° C., or more than 400° C., or more than 550° C. may be heated and used, e.g., to manage thermal losses down the probe body or inner tube. Temperature of the thermal fluid may be anywhere from 0° C. to 550° C., or more than 550° C. Embodiments are also possible in which thermal fluid temperatures of below 0° C. can be implemented.

Embodiments of the console (112, 212) may implement one heater (300) per channel with 4 channels in the console (e.g., two of the ports (119) may be a channel, where one channel is used for cooling and one for heating). In an embodiment, four heaters may be implemented in a console, with the console including four channels and eight ports (each channel capable of cooling or heating). However, it will be understood that many other configurations are possible. Embodiments are also possible in which a set of two ports (119) in a channel are symmetrical in operation. In other words, either both ports may be used for cooling at the same time, or both may be used for heating, if connected.

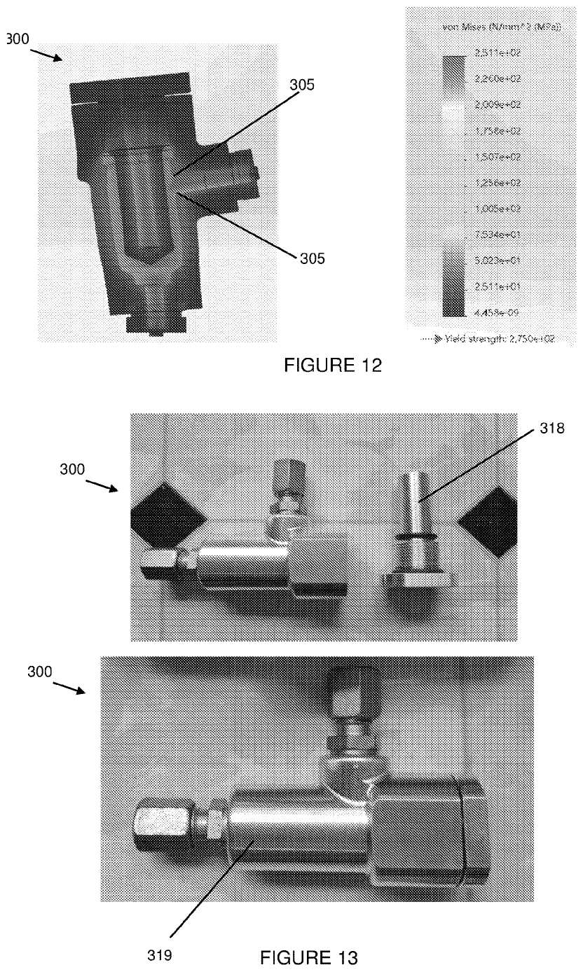

FIG. 12 shows exemplary experiments conducted on the heater (300), with a 40 bar simulated pressure vessel. Higher material stresses were observed only at non-critical positions (e.g., designated with the reference numeral (305)). Physical examples of the heater (300) and its heating element rod or shaft (318) are shown in the photographs of FIG. 13.

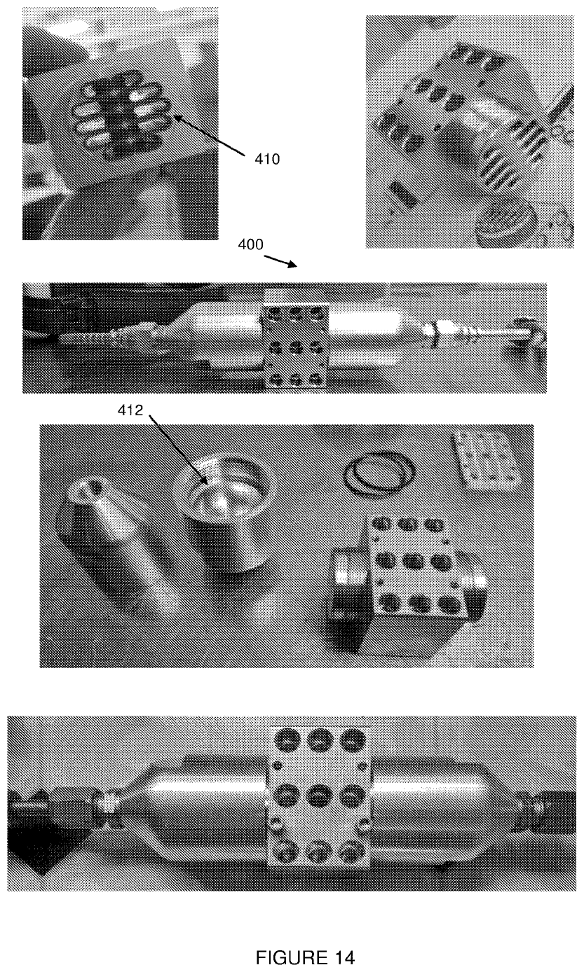

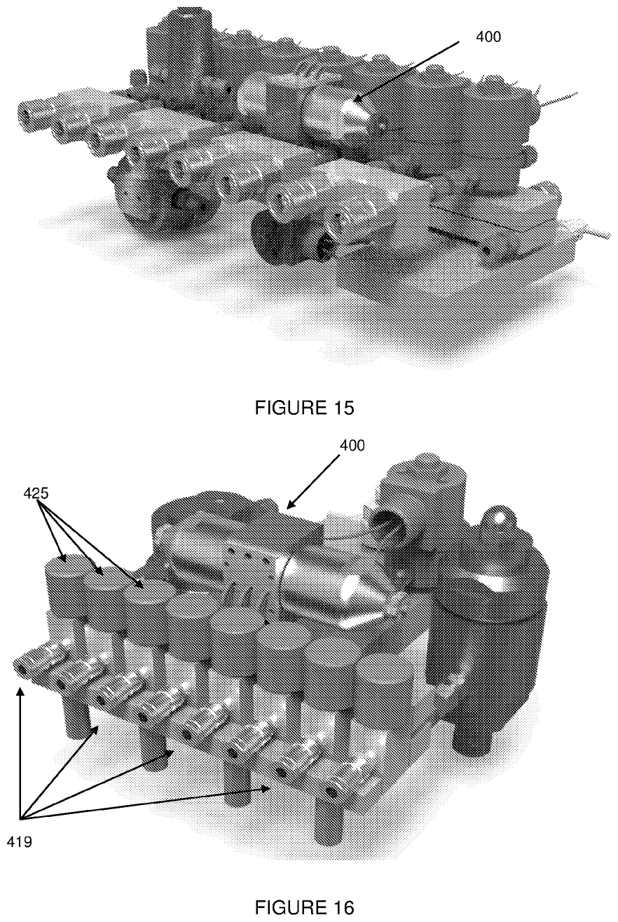

FIG. 14 shows photographs of another exemplary embodiment of a heater (400) with internal heating elements or fins (410), for heat transfer to the thermal fluid, that may be used in embodiments of the present disclosure. Thermal fluid may pass through these fins inside a heating chamber (412), and the fins may for example be electrically heated. An example embodiment of internal components of an exemplary cryoablation system's console is shown in the renderings of FIGS. 15 and 16. In this exemplary embodiment, four channels may be provided and each of these four channels may provide heating or cooling. Two ports may be used per channel for heating. Two ports may be used per channel for cooling. Each channel may be switched between heating or cooling, e.g., by way of a changeover valve. It will be appreciated that the present embodiments may include or implement one or more features of the other embodiments of the present disclosure and vice versa.

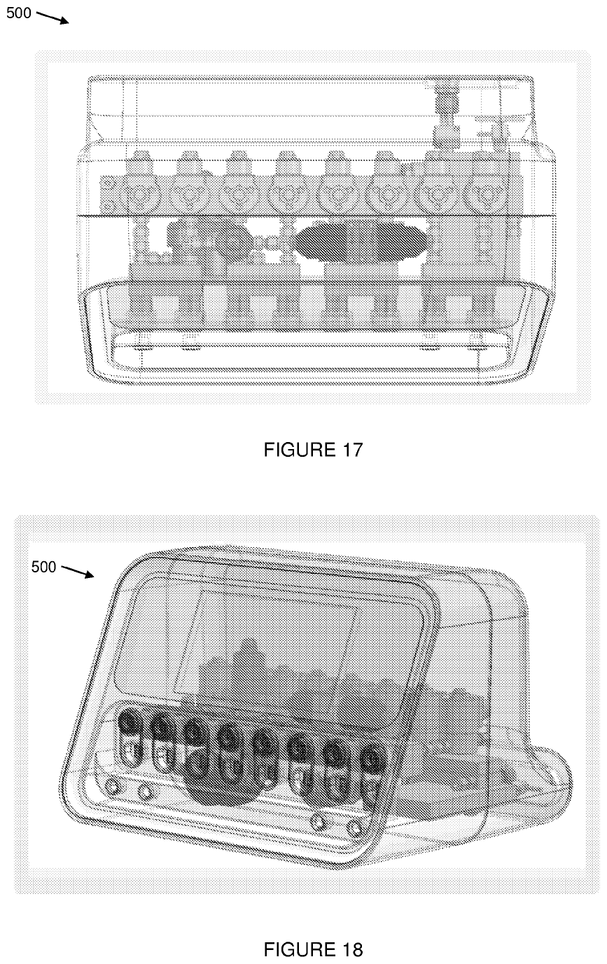

It is envisaged that a single heater (400) may optionally be used for all four channels, e.g., for four channels, including all eight ports (419). All four channels (8 ports) may be used for cooling. However, as mentioned above, it may be preferable to implement four of the smaller heaters (300), e.g., one of these heaters (300) per channel. Solenoid valves (425) may be used to control fluid flow to each of the ports, e.g., controlled by the controller of the console. One or more changeover valves (optionally automatically actuated by the controller) may also be implemented, as explained with reference to FIGS. 6 and 7. A manifold may also be used, to direct flow of the first fluid and/or the second fluid (e.g., the heated fluid and the non-heated fluid/cooling fluid for cooling), as the case may be, to the various ports. Renderings of an exemplary embodiment of a console (500) implementing the internal components of FIGS. 16-17 are shown in FIGS. 17-18. As before, it will be appreciated that these embodiments of the present disclosure may include or implement one or more features of the other embodiments described, and vice versa. For example, each channel may implement a single heating element (300) or heater, instead of using the finned heater (400) for all four channels.

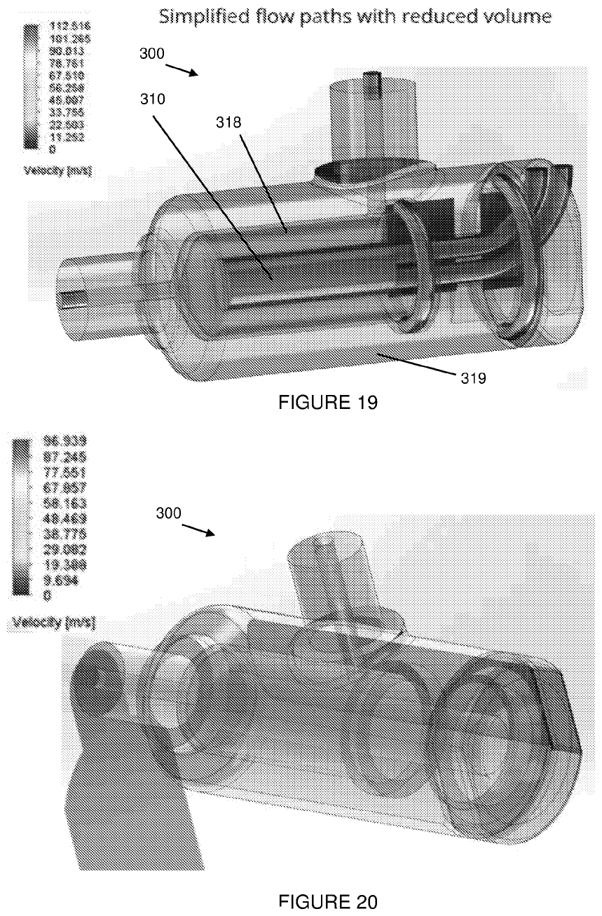

Further experiments and simulations on the heater (300) are shown in FIGS. 19 and 20, demonstrating a simplified fluid flow path, and a reduced volume of its heating chamber (i.e., in the tolerance or void between the outer body (319) and the shaft/rod (318) for the heating element (310)).

Systems and methods of the present disclosure may thaw the ice-ball (e.g., in tissue surrounding the probe needle) faster than using currently available systems or methods. The heated fluid may facilitate thawing/heating of the probe and/or surrounding ice-ball so as to facilitate the probe to be released from the ice ball in the patient's body.

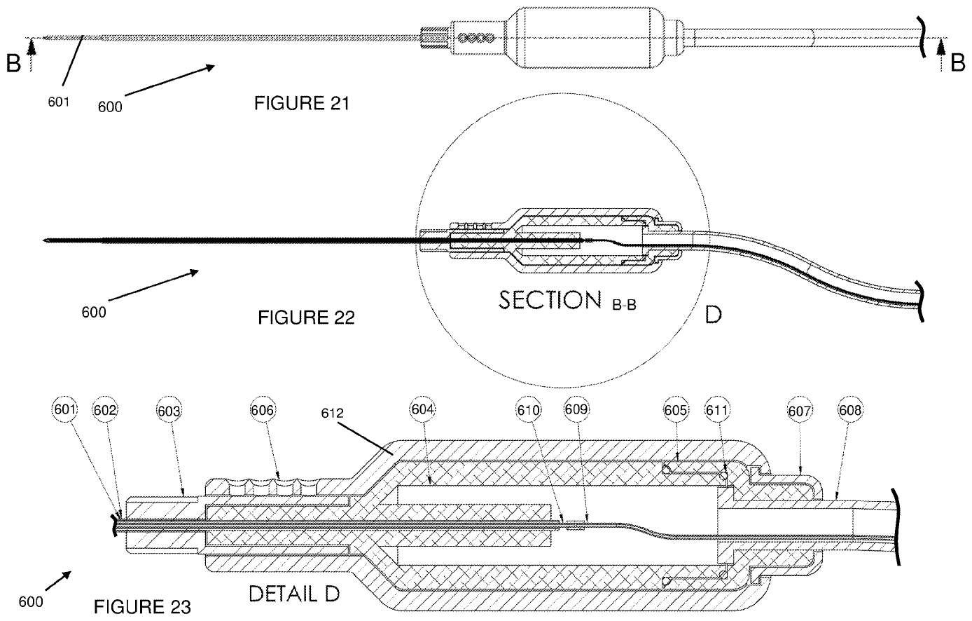

Referring to FIGS. 21-23, an exemplary embodiment of a probe or cryoprobe (600) is shown. The probe (600) may be used in conjunction with the systems and methods of the present disclosure. The probe (600) may include a needle portion (601) (also termed a probe body) and an outer tube (602) (e.g., over an inner needle body). A grip or handle (603) may be provided, an inner body (604) of the handle or grip may be arranged to locate the needle (601) securely within the handle or grip (603). Optionally this grip (603) may be separate from a main probe handle portion (612), or it may be integrated into a single housing or structure to contain the components of the probe (600). A cap, also termed a cap body (605) may be provided to close off a proximal portion of the probe (600). A plurality of adjustment notches (606) may optionally be provided in the probe handle portion (612), e.g., for an adjustable heat-insulating sleeve (e.g., FIG. 5) that may be arranged to slide over the needle so as to adjust a distal portion of the needle that forms the ice-ball, and to adjust an intermediate portion of the needle which is insulated so as not to freeze. A secondary cap or closure (607) may also be provided to secure the inner body (604) to the handle or grip of the probe (600), providing a substantially fluid tight seal, e.g., by way of O-ring seals (611) or other types of seals. An “umbilical” (608) may be arranged to connect the probe to the console of the present disclosure. The “umbilical” may also have heat-insulating material around it, or it may be insulated so as to inhibit heat transfer to the environment, hence increasing the efficiency by which heat is transferred to, or from the probe in use. Heat, or cold isolation or insulation may be provided around the “umbilical”. The “umbilical” or “umbilical cord/tether” may also be termed thermal fluid tubing, optionally also including signal cable(s) or wires, or data communication cable(s).

The “umbilical” may have insulation of the internal components (electrical and fluid tubing) for multiple reasons, including, but not limited to:
Electrical Safety;
User Safety from burns or freezing;
In order to Maintain flexibility;
To inhibit heat loss(es) during thawing; and
Heat absorption during freezing.

The heat insulating material may be selected or composite manufacturing may be used, and a specific material or a combination of materials may be used so as to be capable of handling both high temperatures and cryogenic temperatures (i.e., substantially low temperatures close to 0° C., or less than 0° C.). This may further result in reduced gas usage during a procedure, resulting in energy savings for the user and/or patient.

Still referring to Referring to FIGS. 21-23, a first thermal fluid tube (609) may be provided, e.g., to supply argon or other thermal fluid from the console to the probe (600), e.g., connecting an inlet of the probe body/needle body (601) to the fluid supply console (e.g., 112, 212, 500). An inner tube (610) may be provided so as to extend inside the needle body (also referred to as the probe body (18) with reference to FIG. 3). The inner tube (610) may be similar to the inner tube (24) described above. This may also be termed an inner argon tube, e.g., when argon is used as thermal cooling fluid. It will be appreciated that the embodiment described with reference to FIGS. 21-23 may include or implement one or more features of the other embodiments of the present disclosure and vice versa.

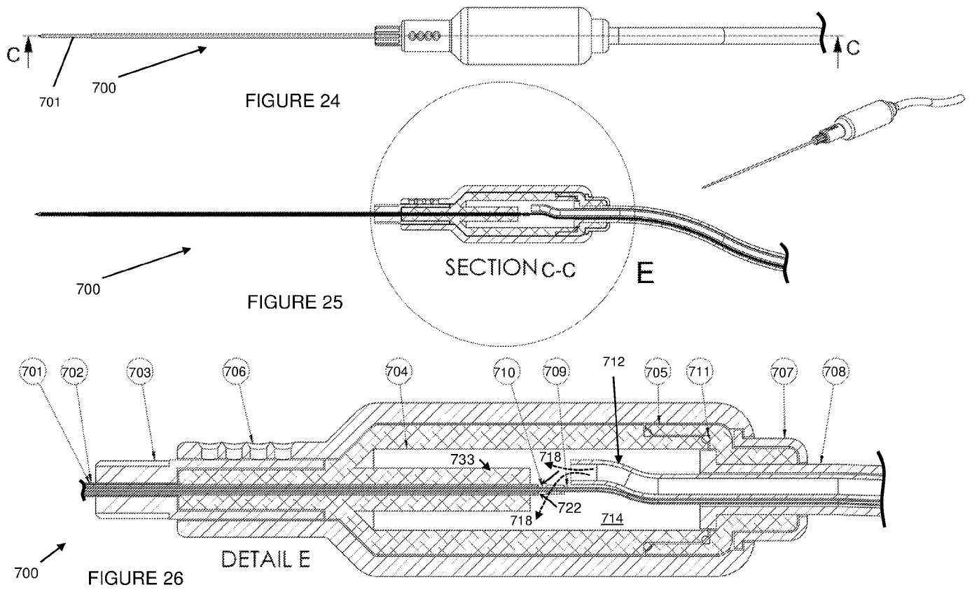

Another embodiment of a probe or cryoprobe (700) is shown in FIGS. 24-26. The probe (700) may be used in conjunction with the systems and methods of the present disclosure. The needle (701); outer tube (702); grip (703); inner body (704); cap (705); adjustment notches (706); secondary cap (707); umbilical, tubing and/or cable (708); first thermal tube (709) e.g., for argon or first thermal fluid; inner tube (710); and seals (711) may be similar to the components described above with reference to FIGS. 21-23. However, in the present embodiment of the probe (700), a heating fluid tube (712) may be provided, e.g., to receive heated thermal fluid from the console (112, 212, 500), such as heated argon, or heated air, or another heated fluid. The heating fluid tube or conduit may be connected to a relevant one of the console ports (e.g., 119) for providing thermal fluid that has been heated by the heating element of the heater (e.g., 114, 214, 300, 400, as the case may be). In other words, a first fluid supply conduit (710) may be provided for a first thermal fluid (e.g., pressurized argon), and a second fluid supply conduit (712) may be provided for a second thermal fluid (e.g., heated argon or heated air, as the case may be). It will be appreciated that the present embodiment may include or implement one or more features of the other embodiments of the present disclosure and vice versa.

Heated thermal fluid (e.g., heater air) may flow from the console through the second fluid supply conduit (712), presently a tube with a larger diameter, and the heated thermal fluid may be expelled or introduced (see directional arrows (718) in broken lines in FIG. 26) into a central heat transfer chamber (714) of the probe (700). This heated thermal fluid may cause heat transfer, by conduction and/or convection, to the inner body (704) of the probe, and to the probe body in general. The inner body (704) of the probe (700) may be manufactured from a heat-conducting material (e.g., a metal) so as to facilitate heat transfer. Heat may be conducted by the inner body (704) (for example in a region (733) that holds the cryo-needle's or probe body's proximal end) toward the needle body (701) (also termed the probe body (e.g. 18) in FIG. 3). The proximal end of the needle body/probe body may be held in this region (733) of the inner body (704) of the probe (700). This heat transfer from the heated thermal fluid may heat up the cryo-needle by conduction.

Moreover, as described above with reference to FIG. 3, the probe body (701, 18) may have an outlet (22 in FIG. 3). In a reverse-flow implementation, heated thermal fluid may also be forced into this outlet (22 in FIG. 3) with the outlet effectively acting as an inlet (722) for thermal fluid, in this case heated thermal fluid. As described above, fluid flow in both directions may be implemented, and heated fluid may be pushed through the outlet (722) (acting as an inlet), or heated air may be pulled through the outlet (722), e.g., by applying a negative pressure to the chamber (714) and for example providing heated fluid through the first fluid supply conduit (710). In other words, embodiments may be possible in which heated fluid such as heated air or heated argon may be pushed/forced; or pulled/sucked through the needle body, as the case may be. This may enable heat transfer to the needle or probe body (18, 701) both by convection and conduction, whether for heating or for cooling/freezing.

Adding a second supply line/conduit (712) to the probe assembly (also termed a needle assembly) with a much larger orifice or a larger diameter as compared to the first fluid supply conduit (710) may facilitate more fluid flow and heat transfer with a reduced JT effect. This may be advantageous, since the heat transfer (in the case of heating) may be improved by a lower JT effect, and it may increase the speed at which the needle or probe body can be thawed or heated, which may also increase patient safety, and ease of use for the user/doctor/surgeon. It will be appreciated that the embodiment described with reference to FIGS. 24-26 may include or implement one or more features of the other embodiments of the present disclosure and vice versa.

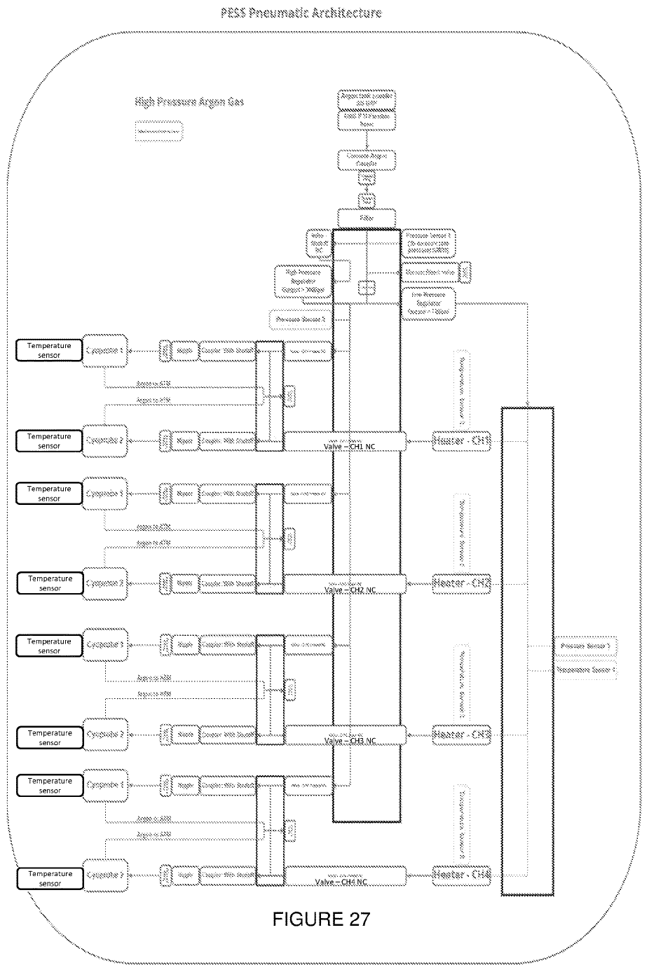

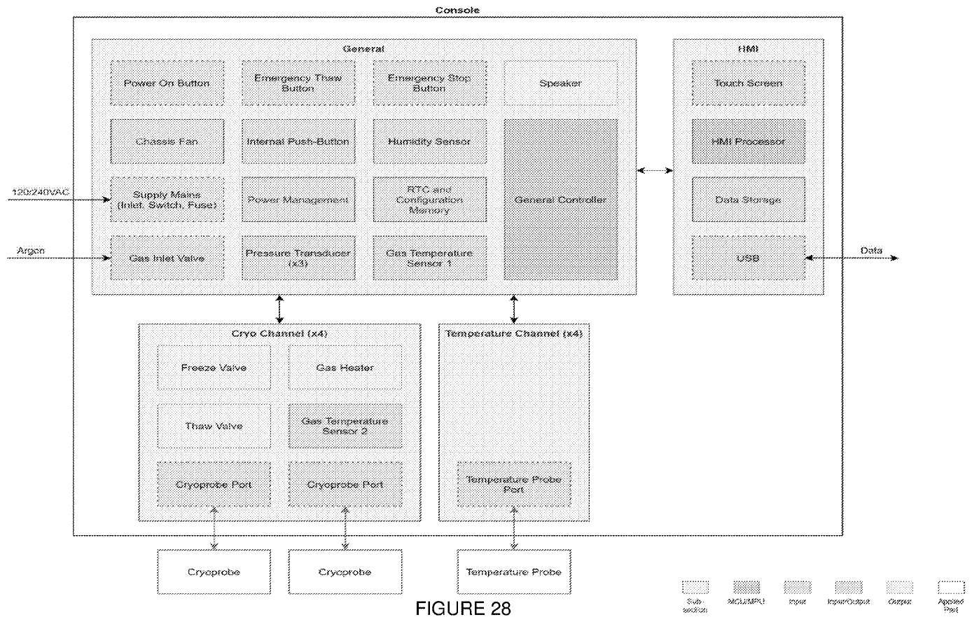

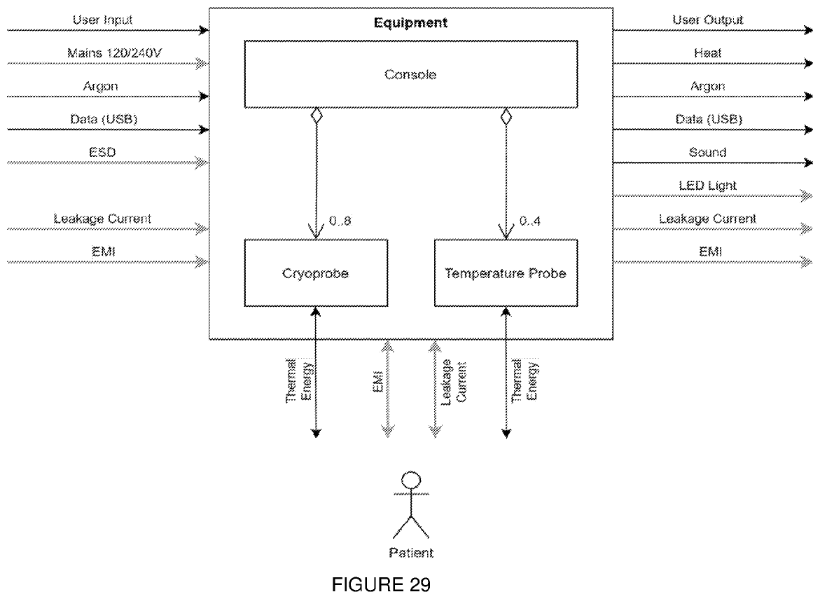

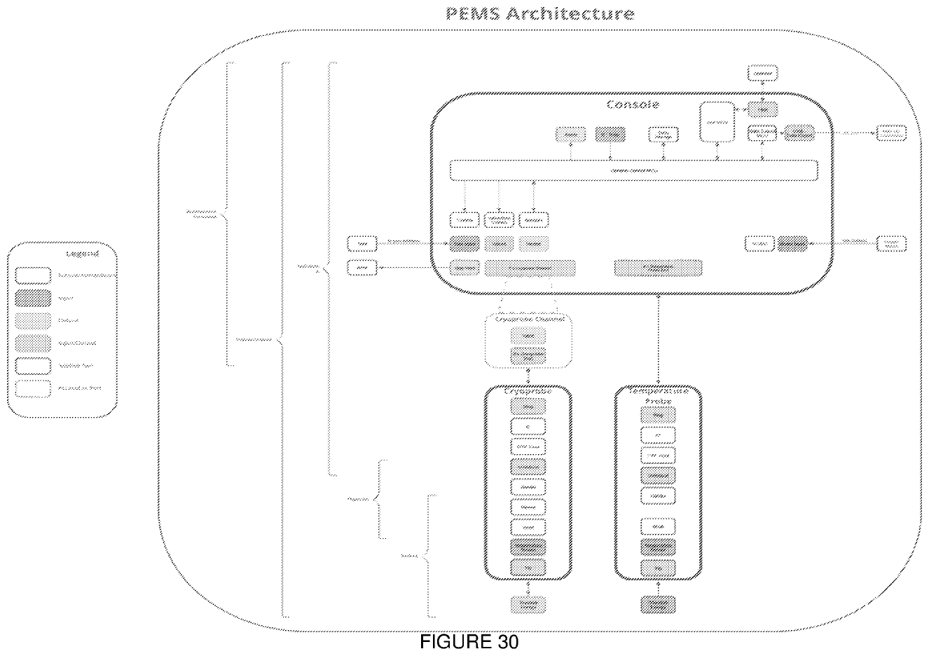

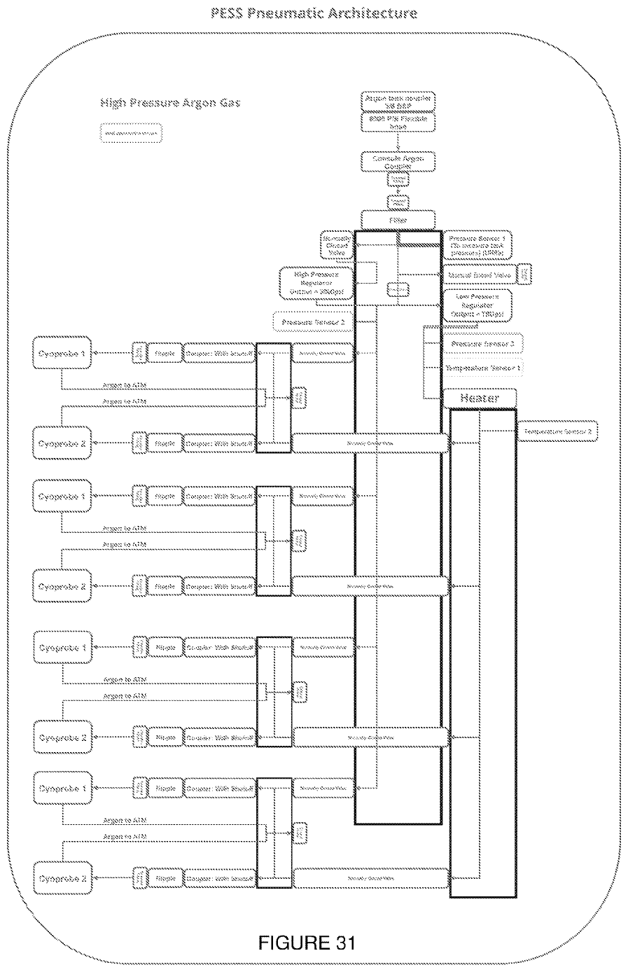

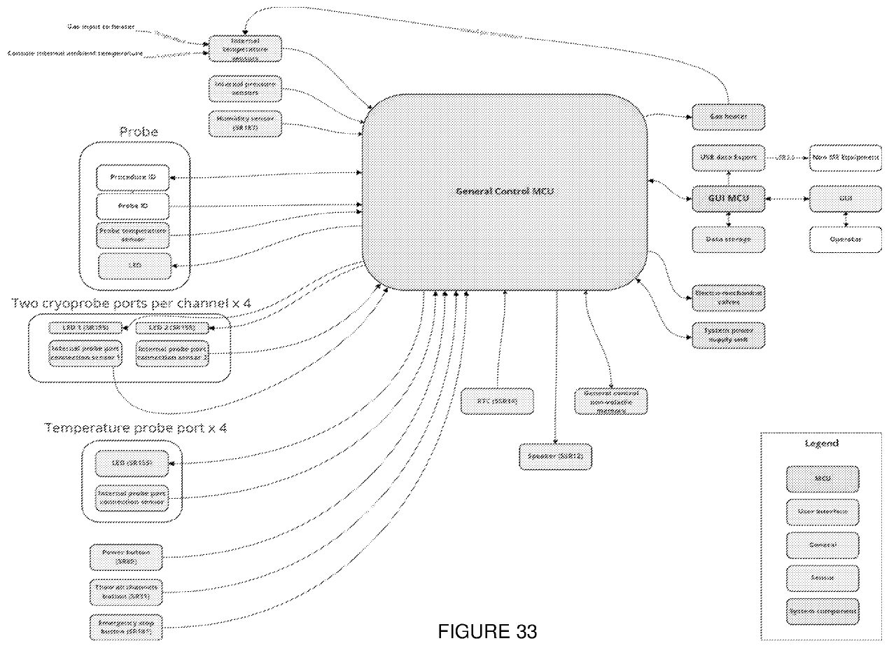

FIGS. 27-31 and 33 are self-explanatory high-level diagrams of exemplary embodiments of the systems of the present disclosure. FIGS. 27-29 are high-level block diagrams of exemplary embodiments of the systems of the present disclosure. FIG. 30 is another high-level block diagram of an exemplary embodiment of a cryoablation system. FIG. 31 is a high-level block diagram of pneumatic architecture and/or fluid flow architecture of an exemplary embodiment of a cryoablation system according to an embodiment of the present disclosure. FIG. 33 is a high-level block diagram of exemplary control software architecture that may be implemented by an embodiment of a cryoablation system of the present disclosure, for example by the processor and/or controller associated with the console. It will be appreciated that these embodiments may include or implement one or more features of the other embodiments of the present disclosure and vice versa.

The following list shows several exemplary, non-limiting, features that may form part of the apparatuses, systems, and methods of the present disclosure.
1. The console may have a physical emergency stop button which stops the flow of argon gas through the console by closing all valves. Pressing the emergency button may activate an alarm.
2. The console may have an emergency thaw button which stops active freezing on all channels and starts active thawing on all active channels.
3. The console may have an information display that provides information to the user such as: Current temperature, freeze power, and procedure time.
4. The UI may give a visual indication if the connected cryoprobe is ready for use.
5. The console may display information in a graph.
6. The console may either not allow a new cryoprobe to be connected to a pressurized channel or may stop the flow of argon on the channel where the console detects a new cryoprobe is being connected.
7. Each connected probe may have a colour element corresponding to the colour indicated on the console UI for that specific probe.
8. The console may have a cryoprobe placement recommendation function indicating the preferred channel to use for the next procedure. The preferred channel may be determined based on the total number of actuation cycles that every channel has undergone.
9. The User Interface (UI) may guide the user through an initial testing procedure.
10. The console may have a thaw function with an active means of heating the cryoprobe needle tip.
11. The following actions may be available for each cryoprobe channel:
   11.1. Start
   11.2. Stop
   11.3. Pause
   11.4. Yes/No button
   There may also be an option to apply one of these actions to all available ports.
12. The console may not allow the use of a particular probe in more than one procedure. The console may not allow the use of a probe where the probe's previous use status cannot be determined. This may facilitate a “single-use” functionality of each probe, which may alleviate risks of infection and/or alleviate or remove the need to clean the probe after use, since it can be disposed. The console may inhibit or prevent user(s) from re-using a probe or needle, since it may track a unique identifier associated with the probe, and store a record indicating that that probe has been used, and warn or prevent the user from re-using that same probe/needle again.
13. The console may display the current Argon working pressure in psi or in Pascal.
14. The console may have a freeze power function that may provide an adjustable measure of the gas delivery to a chosen cryoprobe channel as a percentage of full working pressure. This may be implemented as a duty cycle with a period of 10 seconds. For example: 80% pressure may be implemented as full working pressure applied for 8 seconds and no pressure for 2 seconds, repeat.
15. The console may be able to automatically detect a cryoprobe after the probe has been connected to the console during procedure mode and add the new probe to the list of cryoprobes available for the procedure.
16. The console may be able to generate and display a procedure report. The report may contain:
   16.1 Cryoprobe freeze temperature for each freeze cycle.
   16.2 Cryoprobe freeze time for each freeze cycle.
   16.3 Cryoprobe thaw time for each thaw cycle.
   16.4 Cryoprobe freeze power applied for each freeze cycle.
   16.5 Working gas pressure at the start and end of the procedure.
   16.6 ID numbers of all probes used during the procedure.
   16.7 Date & time of the procedure
   16.8 Cryoprobe test results.
   16.9 Console ID.
17. The console may generate and display a unique number for each procedure.
18. The console may have a means of producing an audible alarm.
19. The console may have a means of displaying an alarm state other than the touch display and may be able to produce both red and orange/yellow light respectively, depending on the alarm condition.
20. The UI may give the user feedback about its alarm state and how to resolve the reason for the alarm (e.g., sensed temperature or sensed pressure exceeds or is less than a threshold). One or more pressure sensor(s) may also be implemented at various locations in the system (e.g., in the probe, piping or at the console), and pressure sensing signals may be received and analysed by the console.

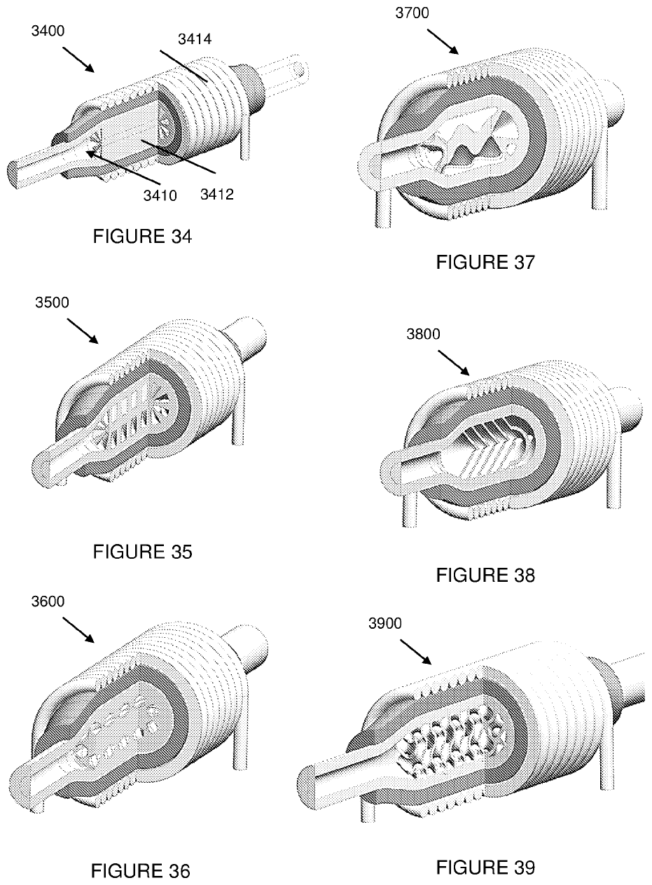

In FIGS. 34-39 are shown a number of exemplary heating elements (3400, 3500, 3600, 3700, 3800, 3900) that may be implemented by aspects of the present disclosure. Each of these heating elements may for example have an internal void (e.g., 3410) wherethrough the thermal fluid may flow. A body (3412) of the heater may be made from a solid material such as a metal with good thermal conductivity. A plurality of inner baffles may be formed by the solid body which may cause turbulence in the thermal fluid as it flows through the void (3410). The heater may be heated by an electrical coil (3414), e.g., by way of induction, e.g., under control of the controller. The electrical induction may heat up the body (3412) which may transfer heat to the thermal fluid (e.g., argon, air etc.) as it passes through on its way to the cryoprobe. The heating element (e.g., 3400) may be provided by the fluid supply console. Optionally a plurality of these heating elements may be implemented, e.g., one for each of a plurality of channels or fluid supply paths that connect to a plurality of probes. Various configurations of the baffles are possible as can be seen in the exemplary embodiments of FIGS. 34-39. Optionally the internal baffles may be omitted, so that the thermal fluid passes straight through the middle of the void.

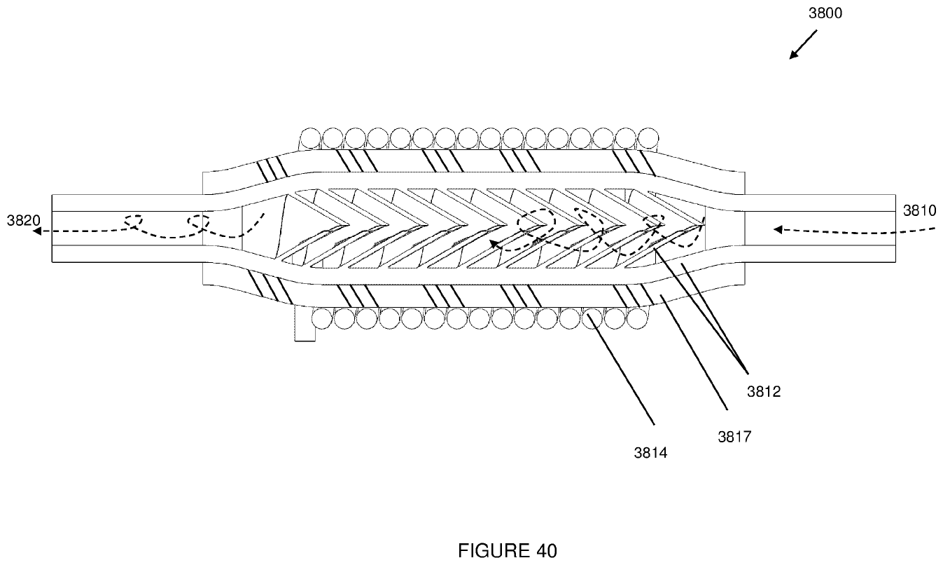

FIG. 40 shows an exemplary sectional view through the heating element (3800) of FIG. 38. Cold or relatively cold thermal fluid (e.g., Argon) may enter the heating element at (3810). The internal baffles may cause turbulence in the thermal fluid, causing heat transfer from the heated core or body (3812) which may be heated up via induction by the coil (3814). The heating element may be insulated by an insulating layer (3817) to inhibit heat losses. Heated thermal fluid (such as Argon) may then exit the heating element (3800) e.g., at (3820) on its way to further components of the system, e.g., toward the cryoprobe. It will be appreciated that the other heating elements may function in a similar way.

These heaters or heating elements may be provided in the console (e.g., 112 in FIG. 1). The heater may also be referred to as a miniature heater. One heater may be provided for a channel, e.g., serving 2 cryoprobes. The heater body may function as a pressure vessel. A surface area for thermal transfer may be maximised, e.g., by way of the baffles. Maximum turbulence may be implemented to the thermal fluid, so as to disturb a boundary layer for improved heat transfer. Variable temperature control may be implemented by the controller. The system may provide a fast ramp up at maximum power. Features of the present disclosure may include duty cycle controlled output temperature of the thermal fluid (e.g., by the controller). Ice-ball size control may also be implemented, using additional heated argon injection instead of switching the high pressure freezing gas on and off, improving solenoid lifetime (e.g., alleviating the need for constant switching of solenoid valves that control thermal fluid flow). The heating system may run at a reduced pressure (e.g., around 25 bar). Optionally, an additional argon (or another type of thermal fluid) cylinder may be used for heating purposes. This may minimise gas wastage (Freeze can e.g., only deplete a cylinder from 6000 psi down to 3000 psi, the rest of the gas is typically discarded). 3000 psi tanks can be connected to the heating system, allowing usage down to <500 psi. An induction heating mechanism may be implemented by aspects of the present disclosure.

In the present embodiments of FIGS. 34-40, the heater body and element may be one and the same. These embodiments may also alleviate the need for pressure seals. The heater may be integrated straight into existing high pressure systems. The heater may include a heater core and an insulator, such as an aerogel blanket or similar. This insulator (e.g., 3817) may separate the heater core/body from the induction coil. It may also isolate the heater from the console interior, reducing heating effects and increasing efficiency by keeping the heat inside. The insulator may also electrically isolate the induction/heating core from the induction coil. It will be appreciated that the embodiments described with reference to FIGS. 34-40 may include or implement one or more features of the other embodiments of the present disclosure and vice versa.

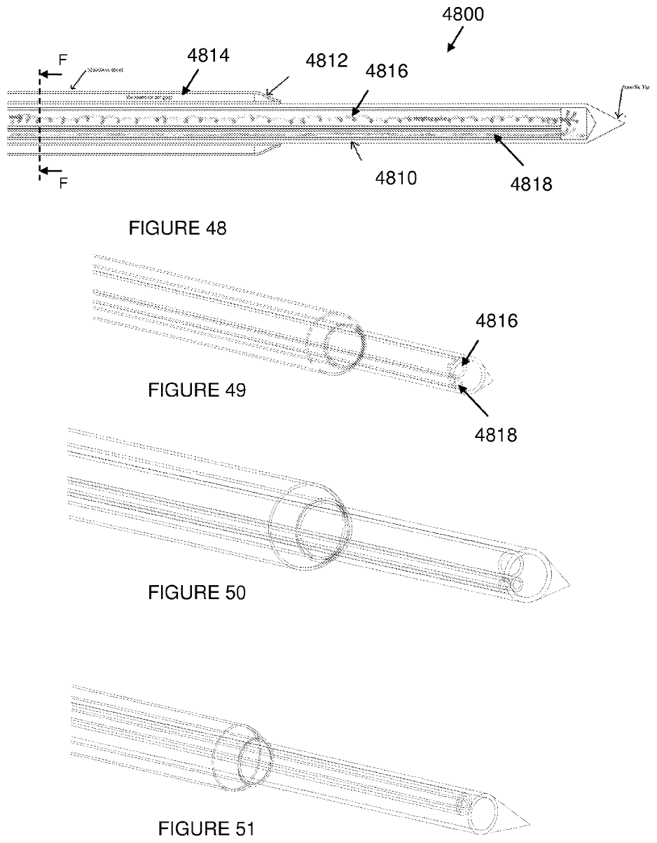

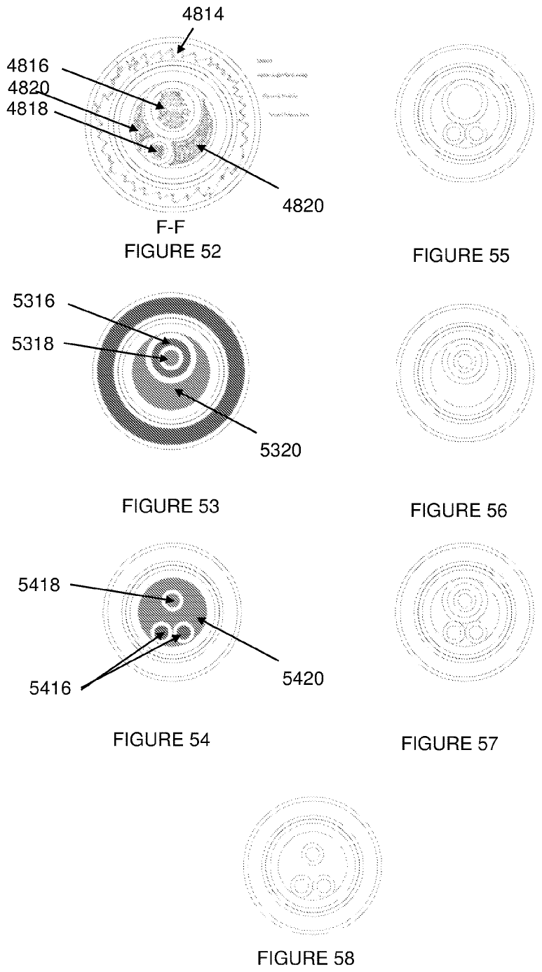

Turning to FIG. 48, there is shown details of an exemplary probe (4800) and its tip according to aspects of the present disclosure. A body (4810) of the probe may also be termed a needle body, and it may e.g., be made from stainless steel. An adjustable insulating sleeve (4812) may be provided so as to control a size of an ice-ball formed, e.g., inside a human body, by the needle. An air gap or vacuum (4814) may be provided in this sleeve, e.g., to facilitate insulation. The sleeve may e.g., also be made from stainless steel or another strong material. Heated thermal fluid may enter the probe through a first fluid path (4816). Thermal fluid for cooling may, on the other hand, enter the probe through a second fluid path (4818) (e.g., see FIG. 49). An exemplary sectional view through the probe is illustrated in FIG. 52 (taken along line F-F in FIG. 48). The vacuum or insulation (4814) is shown diagrammatically in FIG. 52, as well as the heated thermal fluid (4816) thermal fluid for cooling (4816). A return, outlet or exhaust fluid path (4820) may also be provided.

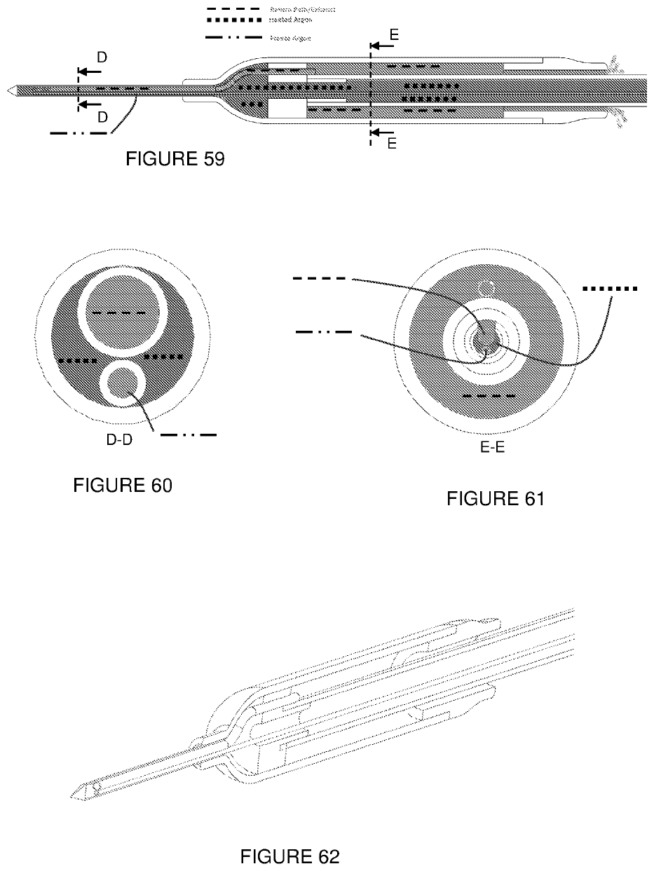

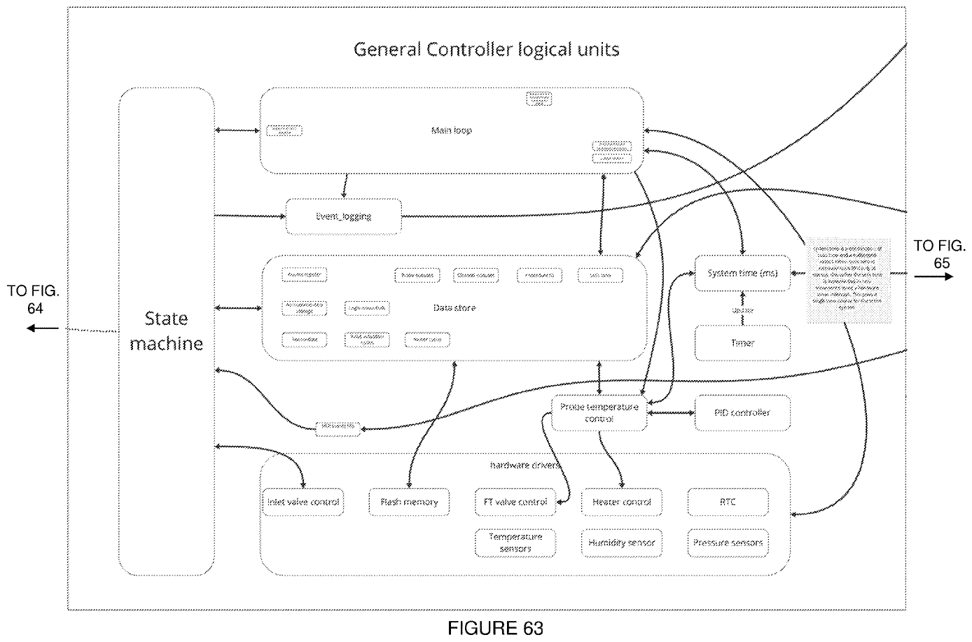

Different arrangements of the outlet or exhaust fluid path (5320, 5420) are shown in FIGS. 53-54. Different arrangements of the inlet or heating fluid path (5316, 5416) are shown in FIGS. 53-54. Different arrangements of the inlet or cooling fluid path (5318, 5418) are shown in FIGS. 53-54. It will be appreciated that many other configurations are possible, as shown in FIGS. 55-62. The needle body may provide a fluid/gas return path (Exhaust). An insulator sleeve (or a vacuum) may be provided for insulation. The sleeve may be adjustable along its length, e.g., so as to be capable of controlling an ice-ball size. A freezing tube may supply argon for implementing the Joule Thomson Freezing effect. A minimal orifice diameter may be selected so as to increase pressure differential for JT effect. One or more thaw tube/s may supply heated argon for thawing or heating. Multiple arrangements are possible, and a larger diameter tube may be used for increased thermal energy transfer at reduced pressure. It will be appreciated that the embodiments described with reference to FIGS. 48-62 may include or implement one or more features of the other embodiments of the present disclosure and vice versa.

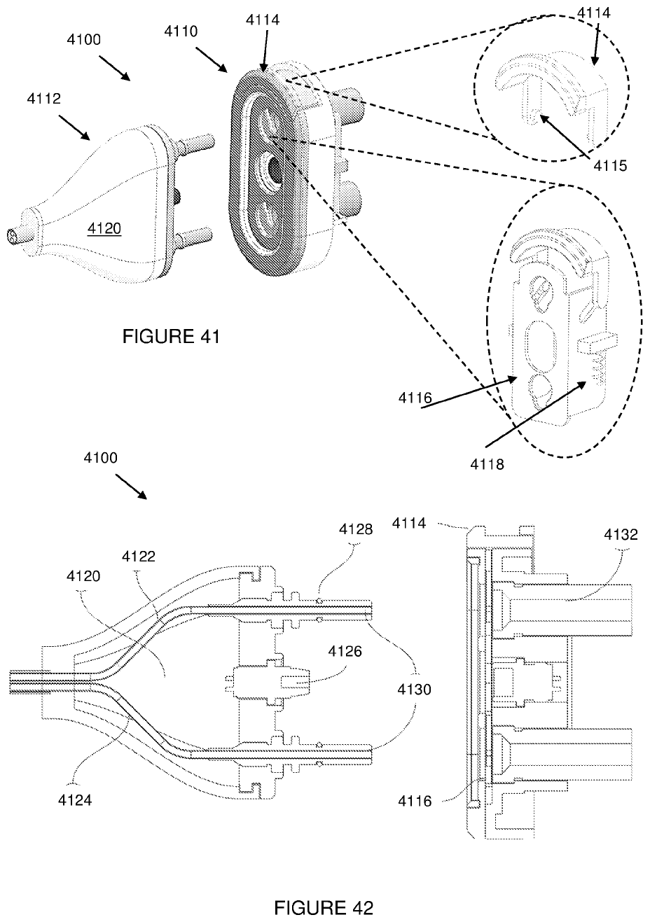

Turning now to FIGS. 41-44, there is shown an exemplary embodiment of a connector (4100) or coupler system that may be used to connect a thermal fluid conduit (e.g., going to the probe) to the fluid supply console. At the console, a socket (4110) may be provided. A plug (4112) or connecting device may be provided to connect thermal fluid to the socket. The plug may, e.g., have two prongs. FIG. 41 also shows enlarged views of an exemplary button and locking device that may be implemented. The locking device may have a button (4114) which may move a locking plate (4116) to lock or unlock the plug or connector from the socket. The button may e.g., be spring loaded by a spring (4118). A plug body (4120) may provide for a handle with internal venting chamber and an exhaust line. The present embodiment may be referred to as a cryoprobe connection to console with a linearly actuated button (4114). A heated (preferably low pressure) line (4122) or tube may be provided by the plug (4120). A cold (preferably high pressure) line or tube (4124) may also be provided by the plug (4120). A circular connector (4126) for a thermocouple or other thermal sensor may also be provided. The locking plate (4116) may be pushed by a user to release the plug or connector therefrom. An O-ring seal (4128) may facilitate a fluid tight coupling. Once the locking plate is moved to release the plug, a sensor may detect this electrically, and the controller may receive a signal indicating that a plug has been disconnected. Once this happens, the controller may actuate an automatic shut-off valve (e.g., a solenoid valve) to stop the flow of thermal fluid.

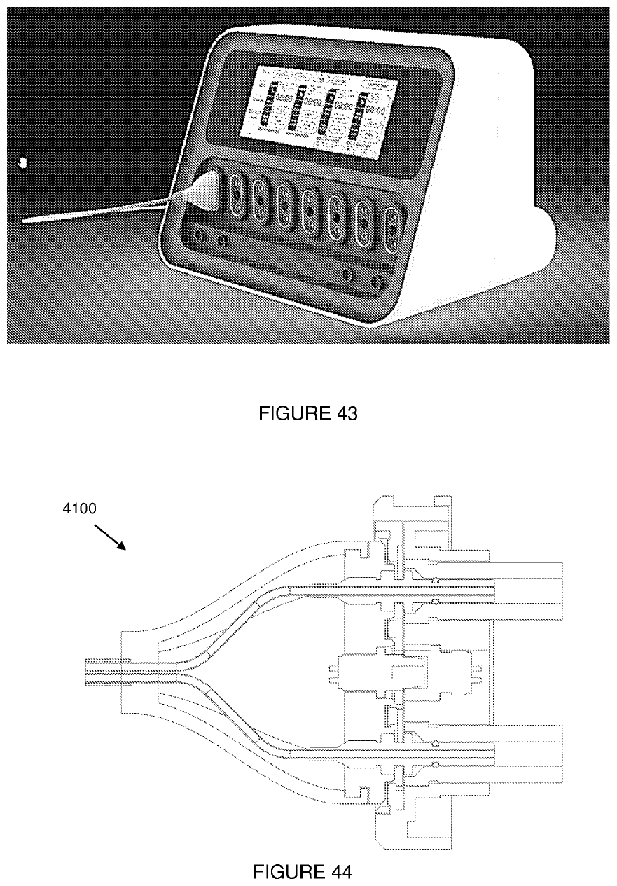

A secondary spring (*not shown) may be provided on the button (4114). A male gas coupler (nipple) (4130) may be provided on a probe side of the coupler (4100). A corresponding female gas receiver (4132) may be provided on the console side (i.e., at the socket (4110)). The button may be actuated by pushing the button forward (i.e., to the right in FIG. 41), which pushes a pin (4115) (or pins) forward and this may cam or slide the plate (4116) down to open it. The plate may then disengage from the male gas coupler allowing the connector's handle (4120) to be pulled out. The pin(s) (4115) on the button may drive linear movement of the locking plate (4116) up or down. A keyhole on the plate may lock onto the male gas coupler. An O-ring seal may be provided between the male gas coupler and female gas receiver. The connector handle (4120) may form a cavity or chamber therein for exhaust gas or exhaust thermal fluid. The male gas coupler may slide into the female gas receiver. An exemplary embodiment of a console having a plug or fluid connector connected to a socket is shown in FIG. 43. The line connecting the plug or connector to the probe may be termed an “umbilical”. One or more automatic fluid closing valves may be provided in the console. The fluid closing valve(s) may implement one or more solenoids or other actuators controlled by the controller. The probe is omitted in this Figure. It will be appreciated that the embodiments described with reference to FIGS. 41-44 may include or implement one or more features of the other embodiments of the present disclosure and vice versa.

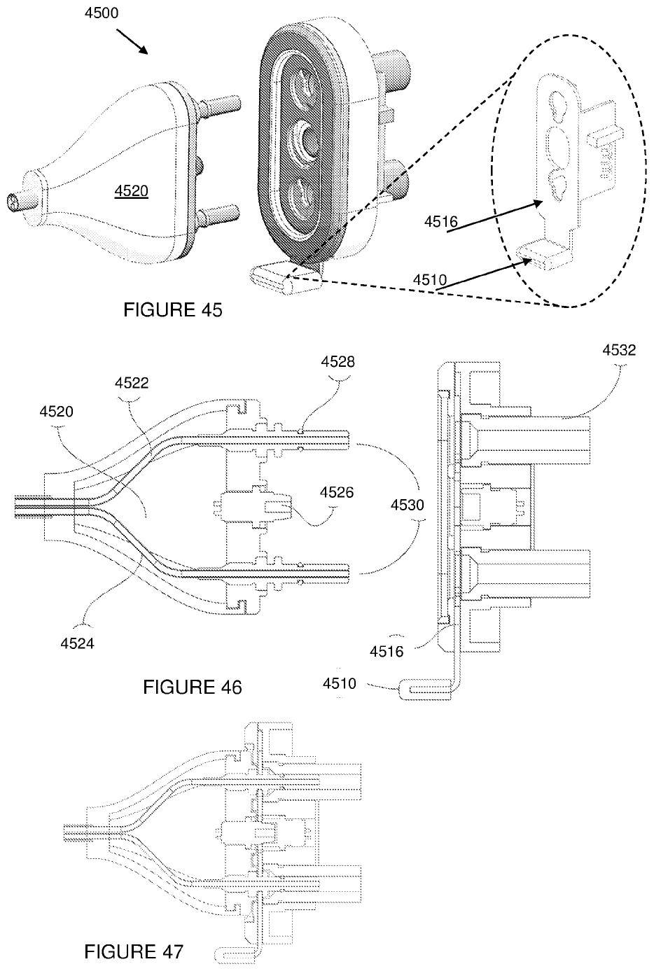

An alternative embodiment of a connector or coupler system (4500) is illustrated in FIGS. 45-47. The present embodiment (4500) is similar to the embodiment described above with reference to FIGS. 41-44. However, in the present embodiment, a linearly actuated lever or tab (4510) may be used to actuate the locking plate (4516). As before, the locking plate may lock or unlock the fluid connector or fluid coupler between the probe and the console of the various embodiments of the present disclosure. The present embodiment may be referred to as a cryoprobe connection to console with a linearly actuated lever. A heated (e.g., low pressure) line (4522) may be provided. A cold (e.g., high pressure) line (4524) may also be provided. The handle (4520) with venting chamber and exhaust line may be similar to that of FIG. 41. A circular connector (4526) for a thermocouple or other sensor may be provided. The locking plate may be slid down to release it, so that the connector can be removed. As before, an O-ring seal (4528) may be implemented. The lever (4510), tab or handle may extend from the locking plate. As before a male gas coupler (nipple) (4530) may be provided on a probe side of the connector system (4500). A female gas receiver (4532) or socket may be provided on the console side. The handle or lever (4510) may be actuated by pulling down, which slides the plate down. The plate then disengages from the male gas coupler allowing the connector (4520) or plug's handle to be pulled out. A keyhole on the plate may lock onto the male gas coupler (4528). The O-ring seal may be between the male gas coupler and female gas receiver. The connector or plug's handle may form a cavity or chamber therein for exhaust fluid, and the male gas coupler may operatively slide into the corresponding female gas receiver. It will be appreciated that the embodiments described with reference to FIGS. 45-47 may include or implement one or more features of the other embodiments of the present disclosure and vice versa.

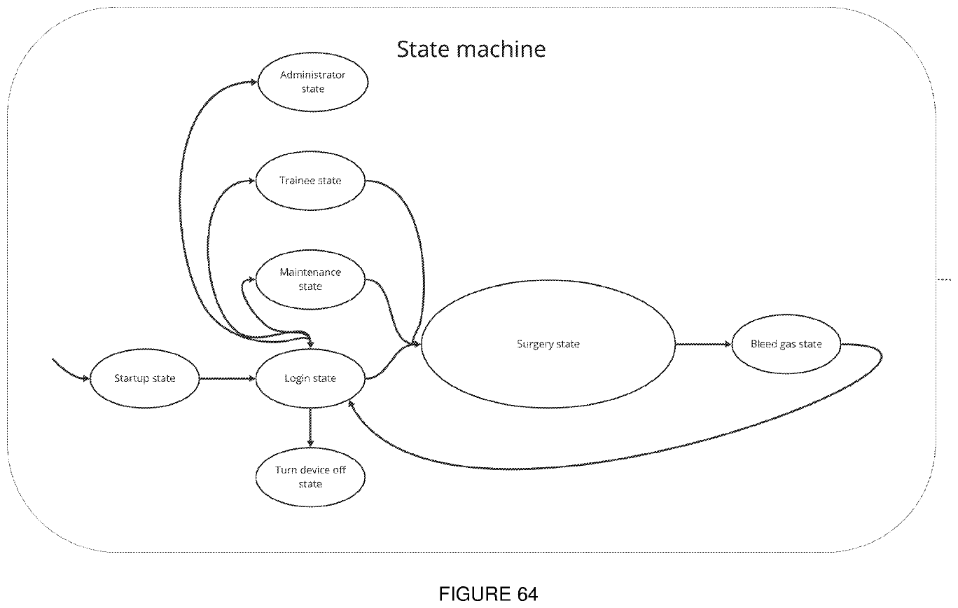

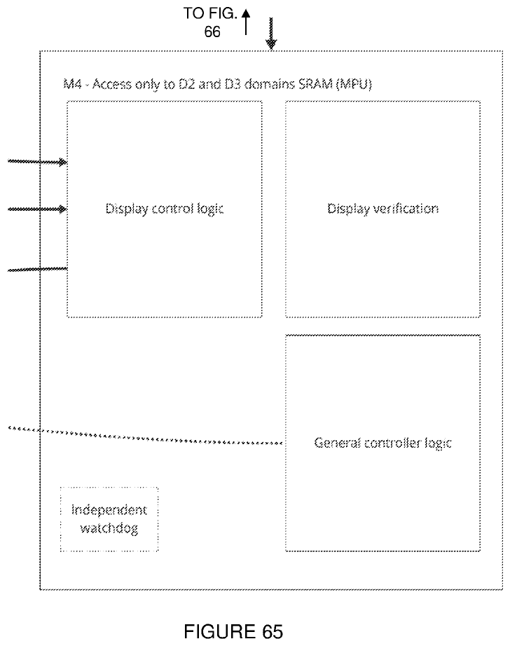

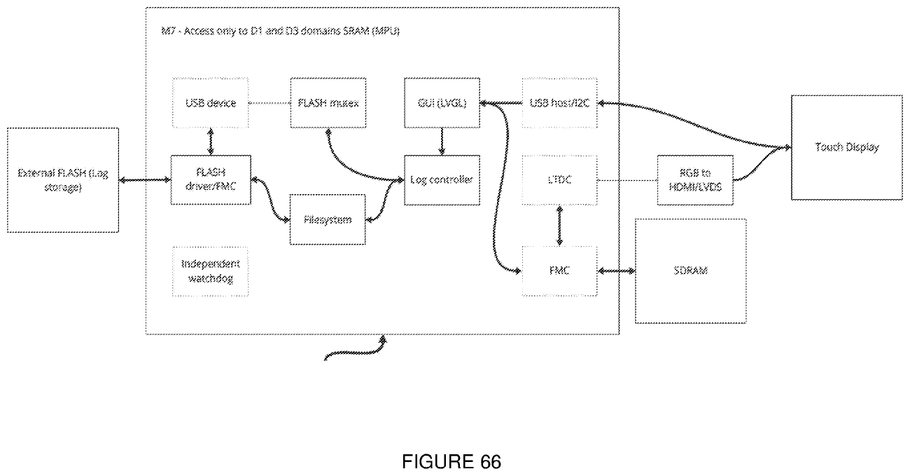

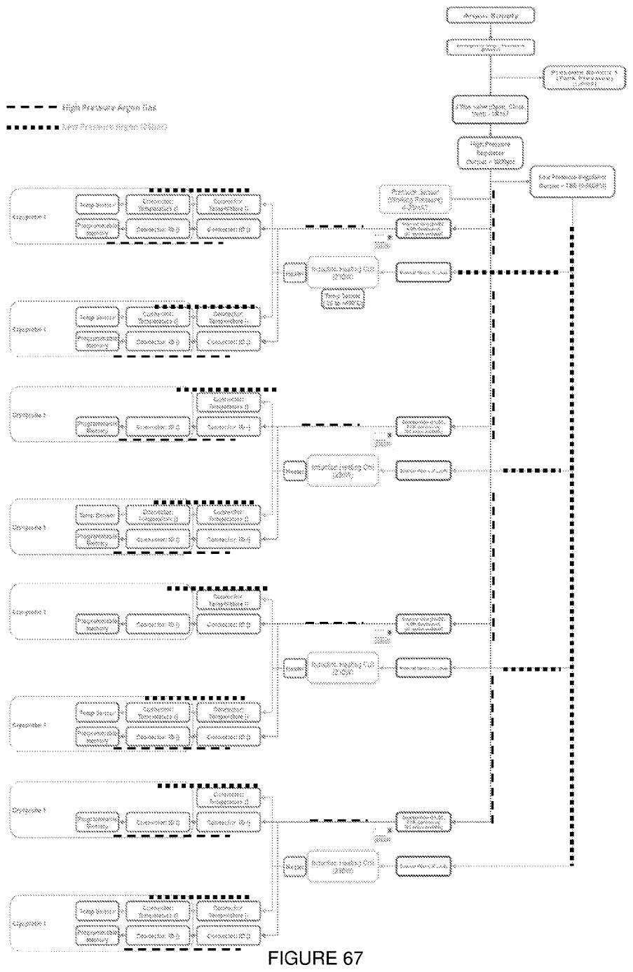

FIGS. 63-67 are self-explanatory high-level block diagrams illustrating exemplary components of cryoablation systems according to aspects of the present disclosure. One or more features of the system may be implemented by intelligent digital control of the controller of the console. A plurality of heaters may be implemented, e.g., as shown in FIG. 67 and each may be used for a different fluid supply channel, leading to a different probe. Each of the channels and their respective heating elements may be controlled by the controller, e.g., by controlling a voltage or a duty cycle of the heating coil. Temperature sensors in one or more of the probes may provide temperature feedback to the controller which can be digitally processed e.g., so as to more accurately control the temperature at the tip of the probe. A response time of the heating may also be reduced, which may be advantageous, especially in an emergency, when rapid thawing or heating is required of the probe.

A plurality of channels or fluid paths may be provided, and each channel may be controlled separately. In an example embodiment, four pressure channels may be used, and only one or two channels may be used at a time, so as to prolong the life of solenoids being used.

A high pressure fluid channel may be used for cooling, whereas heating may be done via a low pressure channel. This may also be more efficient, seeing as high pressure may be more cumbersome or expensive. A freezing or cooling tube may be provided in each needle or probe, and a heating tube may be provided for each needle or probe.

It may be advantageous to provide a plurality of inductive heaters, instead of a single heater with a plurality of solenoid valves for controlling the flow of heated fluid to the probe(s). The heaters may have a generally lower cost as compared to the solenoid(s). The manifold may facilitate flow of thermal fluid, either for heating or for cooling, as the case may be.

The foregoing description has been presented for the purpose of illustration; it is not intended to be exhaustive or to limit the invention to the precise forms disclosed. Persons skilled in the relevant art can appreciate that many modifications and variations are possible in light of the above disclosure.

The language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based hereon. Accordingly, the disclosure of the embodiments of the invention is intended to be illustrative, but not limiting, of the scope of the invention, which is set forth in the following claims.

Finally, throughout the specification and accompanying claims, unless the context requires otherwise, the word ‘comprise’ or variations such as ‘comprises’ or ‘comprising’ will be understood to imply the inclusion of a stated integer or group of integers but not the exclusion of any other integer or group of integers.

1. A cryoablation system comprising:
a probe which has a hollow body that defines an inlet and an outlet, the inlet configured to receive a thermal fluid for circulation through the hollow body, and the outlet configured to discharge the thermal fluid from the hollow body; and
a fluid supply console which is remote from the probe and in fluid communication with the inlet of the probe, wherein the fluid supply console is arranged, in use, to provide the thermal fluid to the probe to enable heating or cooling by the probe, and wherein the fluid supply console includes a heating element which is configured to selectively heat the thermal fluid before it is provided to the inlet of the probe so as to enable selective heating of the probe in use.

2. The cryoablation system of claim 1, wherein the system includes a controller having a processor and a memory, said memory containing instructions executable by said processor, to execute functions of one or more components of the system.

3. The cryoablation system of claim 2, wherein the fluid supply console includes at least one valve which is operable between a first state and a second state, wherein in the first state the valve is configured to provide the thermal fluid to the probe for cooling, and in the second state the valve is configured to provide the thermal fluid heated by the heating element to the probe.

4. The cryoablation system of claim 3, wherein the controller is operable to automatically select the first state and/or the second state of the valve so as to selectively provide heating or cooling of the probe in use.

5. The cryoablation system of claim 2, wherein the controller is operable to control the heating element so as to selectively heat the thermal fluid.

6. The cryoablation system of claim 1, wherein the fluid supply console is arranged to provide a single type of fluid as thermal fluid to the probe to facilitate cooling by the probe in use.

7. The cryoablation system of claim 6, wherein the heating element is arranged to selectively heat the single type of thermal fluid before it is provided to the probe, so as to selectively heat the probe body.

8. The cryoablation system of claim 1, wherein the fluid supply console is arranged to selectively provide: a first thermal fluid for cooling the probe; and a second thermal fluid for heating the probe.

9. The cryoablation system of claim 8, wherein the heating element of the fluid supply console is arranged to selectively heat the second thermal fluid before it is provided to the inlet of the probe.

10. The cryoablation system of claim 1, wherein the fluid supply console is connected to a pressurized thermal fluid supply.

11. A fluid supply console configured for use in the cryoablation system of claim 1.

12. A probe configured for use in the cryoablation system of claim 1.

13. A method of applying cryoablation treatment, the method comprising:
providing a probe which has a hollow body that defines an inlet and an outlet, the inlet configured to receive a thermal fluid for circulation through the hollow body, and the outlet configured to discharge the thermal fluid from the hollow body;
providing a fluid supply console remotely and in fluid communication with the inlet of the probe;
by the fluid supply console, operatively supplying the thermal fluid to the probe so as to enable heating or cooling by the probe; and
by a heating element of the fluid supply console, selectively heating the thermal fluid before it is provided to the inlet of the probe so as to selectively heat the probe in use.
